(19) United States
(12) Patent Application Publication
(10) Pub. No.: US 2023/0112648 A1
(43) Pub. Date: Apr. 13, 2023

(54) NON-NATURALLY OCCURRING POLYADENYLATION ELEMENTS AND METHODS OF USE THEREOF

(71) Applicant: Homology Medicines, Inc., Bedford, MA (US)

(72) Inventors: Serena Nicole DOLLIVE, Bedford, MA (US); Andrew Pla, Bedford, MA (US)

(21) Appl. No.: 17/932,961

(22) Filed: Sep. 16, 2022

Related U.S. Application Data
(60) Provisional application No. 63/261,322, filed on Sep. 17, 2021.

(51) Int. Cl. C12N 15/67

(52) U.S. Cl. CPC C12N 15/67; C12N 2830/50

(57) ABSTRACT

Provided herein are polynucleotides and vectors comprising non-naturally occurring polyadenylation (polyA) sequences, and methods of making and using these polynucleotides and vectors.

### RELATED APPLICATIONS

This application claims priority to U.S. Provisional Patent Application Ser. No. 63/261,322, filed Sep. 17, 2021, the entire disclosure of which is hereby incorporated herein by reference.

### SEQUENCE LISTING

This application contains a sequence listing which has been submitted electronically in ASCII format and is hereby incorporated by reference in its entirety (said ASCII copy, created on Sep. 15, 2022, is named “HMW-036 Sequence Listing” and is 49,444 bytes in size).

### BACKGROUND

In nature, individual genes have their own unique polyadenylation (polyA) sequence, which signals for the termination of transcription when placed 3′ of a coding sequence. Termination of transcription involves the release of RNA polymerase II from the nascent transcript, cleavage of the nascent transcript, and polyadenylation of the 3′ end of the new transcript. PolyA sequences are also employed in recombinant gene expression cassettes to terminate transcription and facilitate polyadenylation. However, naturally occurring polyA sequences vary greatly in their transcriptional termination efficiency, size, and genetic origin; which, in some instances, can make them unsuitable for use in gene expression vectors, particularly those vectors intended for administered to humans. Therefore, there is a need for novel non-naturally occurring polyA sequences for use in gene expression cassettes to, inter alia, maximize gene expression, optimize size of a cassette or vector, and/or optimize the percentage of a polyA sequence that is derived from a particular species of origin or single human gene.

### SUMMARY

Provided herein are polynucleotides and vectors comprising non-naturally occurring chimeric polyadenylation (polyA) sequences, and methods of making and using these polynucleotides and vectors. The compositions disclosed herein are particularly useful for use in gene therapy vectors (e.g., human gene therapy vectors).

Accordingly, in one aspect the instant disclosure provides a polynucleotide comprising a non-naturally occurring polyadenylation (polyA) sequence, said polynucleotide comprising from 5′ to 3′: a polyA signal that comprises the nucleic acid sequence set forth in SEQ ID NO: 1; a first intervening nucleic acid sequence derived from a naturally occurring polyA sequence of a first gene, wherein said naturally occurring polyA sequence of a first gene comprises a polyA signal, a GT rich region, and a nucleic acid sequence positioned between said polyA signal and said GT rich region, wherein said first intervening nucleic acid sequence comprises a sequence of at least 10 nucleotides in length that is derived from said nucleic acid sequence positioned between said polyA signal and said GT rich region of said naturally occurring polyA sequence of a first gene, and wherein said first intervening nucleic acid sequence comprises 0, 1, 2, or 3 nucleotide modifications relative to the corresponding region of said naturally occurring polyA sequence of a first gene; and a first GT rich nucleic acid sequence derived from a naturally occurring polyA sequence of a second gene, wherein said naturally occurring polyA sequence of a second gene comprises a polyA signal and a GT rich region; wherein said first GT rich nucleic acid sequence comprises a sequence of at least 5 nucleotides in length that is derived from said GT rich region of said naturally occurring polyA sequence of a second gene, wherein said first GT rich nucleic acid sequence comprises 0, 1, or 2 nucleotide modifications relative to the corresponding region of said naturally occurring polyA sequence of a second gene, and wherein said first GT rich nucleic acid sequence is positioned 10-30 nucleotides downstream of said polyA signal that comprises the nucleic acid sequence set forth in SEQ ID NO: 1; and wherein said first gene and said second gene are different.

In some embodiments, said first gene is a non-human gene. In some embodiments, said non-human gene is a viral, bacterial, or non-human mammalian gene. In some embodiments, said first non-human gene is a viral gene. In some embodiments, said viral gene is simian virus 40 (SV40) late gene. In some embodiments, said first intervening nucleic acid sequence derived from a naturally occurring polyA sequence of a first gene comprises the nucleic acid sequence set forth in SEQ ID NO: 4. In some embodiments, said first gene is a human gene.

In some embodiments, said second gene is a non-human gene. In some embodiments, said second gene is a human gene. In some embodiments, said second gene is human growth hormone (HGH). In some embodiments, said first GT rich nucleic acid sequence derived from a naturally occurring polyA sequence of a second gene comprises the nucleic acid sequence set forth in SEQ ID NO: 2.

In some embodiments, said first GT rich nucleic acid sequence is positioned 15-30 nucleotides downstream of said polyA signal that comprises the nucleic acid sequence set forth in SEQ ID NO: 1.

In some embodiments, said polynucleotide is no more than 300, 250, or 200 nucleotides in length.

In some embodiments, said polynucleotide further comprises a second GT rich nucleic acid sequence derived from a naturally occurring polyA sequence of a third gene, wherein said naturally occurring polyA sequence of a third gene comprises a polyA signal and a GT rich region; wherein said second GT rich nucleic acid sequence comprises a nucleic acid sequence of at least 5 nucleotides in length that is derived from said GT rich region of said naturally occurring polyA sequence of a third gene; wherein said second GT rich nucleic acid sequence comprises 0, 1, or 2 nucleotide modifications relative to the corresponding region of said naturally occurring polyA sequence of a third gene; and wherein said second GT rich nucleic acid sequence is positioned 5-100 nucleotides downstream of said first GT rich nucleic acid sequence.

In some embodiments, said third gene is a human gene. In some embodiments, said third gene is HGH. In some embodiments, said second GT rich nucleic acid sequence derived from a naturally occurring polyA sequence of a third gene comprises the nucleic acid sequence set forth in SEQ ID NO: 3. In some embodiments, said third gene is a non-human gene.

In some embodiments, said third gene and said second gene are the same. In some embodiments, said third gene and said second gene are different.

In some embodiments, said polynucleotide further comprises a second intervening nucleic acid sequence derived from a naturally occurring polyA sequence of a fourth gene, wherein said naturally occurring polyA sequence of a fourth gene comprises a first GT rich region, a second GT rich region, and a nucleic acid sequence positioned between said first GT rich region and said second GT rich region, wherein said second intervening nucleic acid sequence comprises a sequence of at least 5 nucleotides in length that is derived from said nucleic acid sequence positioned between said first GT rich region and said second GT rich region of said naturally occurring polyA sequence of a fourth gene, and wherein said second intervening nucleic acid sequence comprises 0, 1, 2, or 3 nucleotide modifications relative to the corresponding region of said naturally occurring polyA sequence of a fourth gene.

In some embodiments, said fourth gene is a human gene. In some embodiments, said fourth gene is a non-human gene. In some embodiments, said non-human gene is a viral, bacterial, or non-human mammalian gene. In some embodiments, said non-human gene is a non-human mammalian gene. In some embodiments, said non-human mammalian gene is bovine growth hormone (BGH) or rabbit beta globin (RBG). In some embodiments, said non-human mammalian gene is RBG. In some embodiments, said second intervening nucleic acid sequence derived from a naturally occurring polyA sequence of a fourth gene comprises the nucleic acid sequence set forth in SEQ ID NO: 5.

In some embodiments, said fourth gene and said first gene are different. In some embodiments, said fourth gene and said first gene are the same.

In some embodiments, said second intervening nucleic acid sequence derived from a naturally occurring polyA sequence of a fourth gene is positioned downstream of said first GT rich nucleic acid sequence and upstream of said second GT rich nucleic acid sequence.

In some embodiments, said polynucleotide further comprises an upstream sequence element derived from a naturally occurring polyA sequence of a fifth gene, wherein said naturally occurring polyA sequence of a fifth gene comprises a polyA signal, a GT rich region, and a nucleic acid sequence positioned immediately upstream of said polyA signal; and wherein said upstream sequence element comprises 1-100 nucleotides derived from said nucleic acid sequence positioned immediately upstream of said polyA signal of said naturally occurring polyA sequence of a fifth gene. In some embodiments, said fifth gene is a human gene. In some embodiments, said fifth gene is a non-human gene.

In some embodiments, said polynucleotide comprises a sequence with at least 95%, 96%, 97%, 98%, 99%, or 100% identity to the sequence set forth in SEQ ID NO: 7.

In some embodiments, said polynucleotide comprises a sequence with 100% identity to the sequence set forth in SEQ ID NO: 7.

In some embodiments, said polynucleotide further comprises a first terminator positioned upstream or downstream of said polyA signal that comprises the nucleic acid sequence set forth in SEQ ID NO: 1.

In some embodiments, said first terminator is selected from the group consisting of a woodchuck hepatitis virus posttranscriptional regulatory element (WPRE), a human C2 pause site element, a SV40 upstream sequence element, an alpha 2 globin pause site element, a human beta globin cotranscriptional cleavage (CoTC) sequence element, and a mouse beta-major globin pause site element.

In some embodiments, said first terminator comprises a human C2 gene pause site element, wherein said human C2 gene pause site element is positioned downstream of said polyA signal that comprises the nucleic acid sequence set forth in SEQ ID NO: 1, or a WPRE, wherein said WPRE is positioned upstream of said polyA signal that comprises the nucleic acid sequence set forth in SEQ ID NO: 1. In some embodiments, said first terminator comprises a nucleic acid sequence of at least 95%, 96%, 97%, 98%, 99%, or 100% identity to the sequence set forth in SEQ ID NO: 8 or 9. In some embodiments, said polynucleotide comprises a second terminator. In some embodiments, said first and said second terminator are different.

In some embodiments, said first terminator comprises a human C2 gene pause site element, wherein said human C2 gene pause site element is positioned downstream of said polyA signal that comprises the nucleic acid sequence set forth in SEQ ID NO: 1, and said second terminator comprises a WPRE, wherein said WPRE is positioned upstream of said polyA signal that comprises the nucleic acid sequence set forth in SEQ ID NO: 1.

In some embodiments, said first terminator comprises a nucleic acid sequence of at least 95%, 96%, 97%, 98%, 99%, or 100% identity to the sequence set forth in SEQ ID NO: 8; and said second terminator comprises a nucleic acid sequence of at least 95%, 96%, 97%, 98%, 99%, or 100% identity to the sequence set forth in SEQ ID NO: 9.

In one aspect, provided herein is a polynucleotide comprising a non-naturally occurring polyadenylation (polyA) sequence, said polynucleotide comprising from 5′ to 3′: an upstream sequence element nucleic acid sequence derived from a naturally occurring polyA sequence of a first gene, wherein said naturally occurring polyA sequence of a first gene comprises a naturally occurring upstream sequence element, a polyA signal, and a GT rich region, wherein said upstream sequence element comprises a functional nucleic acid sequence of said naturally occurring upstream sequence element of said naturally occurring polyA sequence of a first gene, and wherein said upstream sequence element nucleic acid sequence comprises 0, 1, 2, or 3 nucleotide modifications relative to the corresponding region of said naturally occurring polyA sequence of a first gene; a polyA signal that comprises the nucleic acid sequence set forth in SEQ ID NO: 1; a first GT rich nucleic acid sequence derived from a naturally occurring polyA sequence of a second gene, wherein said naturally occurring polyA sequence of a second gene comprises a polyA signal and a GT rich region; wherein said first GT rich nucleic acid sequence comprises a sequence of at least 5 nucleotides in length that is derived from said GT rich region of said naturally occurring polyA sequence of a second gene, wherein said first GT rich nucleic acid sequence comprises 0, 1, or 2 nucleotide modifications relative to the corresponding region of said naturally occurring polyA sequence of a second gene, and wherein said first GT rich nucleic acid sequence is positioned 10-30 nucleotides downstream of said polyA signal that comprises the nucleic acid sequence set forth in SEQ ID NO: 1; and wherein said first gene and said second gene are different.

In some embodiments, said first gene is a non-human gene. In some embodiments, said non-human gene is a viral, bacterial, or non-human mammalian gene. In some embodiments, said non-human gene is a viral gene. In some embodiments, said viral gene is simian virus 40 (SV40) late gene. In some embodiments, said upstream sequence element nucleic acid sequence comprises the nucleic acid sequence set forth in SEQ ID NO: 13. In some embodiments, said upstream sequence element nucleic acid sequence comprises the nucleic acid sequence set forth in SEQ ID NO: 15.

In some embodiments, said first gene is a human gene.

In some embodiments, said second gene is a non-human gene. In some embodiments, said second gene is a human gene. In some embodiments, said human gene is HGH. In some embodiments, said first GT rich nucleic acid sequence derived from a naturally occurring polyA sequence of a second gene comprises the nucleic acid sequence set forth in SEQ ID NO: 2.

In some embodiments, said first GT rich nucleic acid sequence is positioned 15-30 nucleotides downstream of said polyA signal that comprises the nucleic acid sequence set forth in SEQ ID NO: 1.

In some embodiments, said polynucleotide is no more than 300, 250, or 200 nucleotides in length.

In some embodiments, said upstream sequence element nucleic acid sequence is positioned immediately upstream of said polyA signal that comprises the nucleic acid sequence set forth in SEQ ID NO: 1. In some embodiments, said polynucleotide comprises at least two copies of said upstream sequence element nucleic acid sequence. In some embodiments, said two copies of said upstream sequence element nucleic acid sequence are consecutively positioned upstream of said polyA signal that comprises the nucleic acid sequence set forth in SEQ ID NO: 1.

In some embodiments, said polynucleotide further comprises a second GT rich nucleic acid sequence derived from a naturally occurring polyA sequence of a third gene, wherein said naturally occurring polyA sequence of a third gene comprises a polyA signal, a first GT rich region, and a second GT rich region; wherein said second GT rich nucleic acid sequence comprises a sequence of at least 5 nucleotides in length that is derived from said second GT rich region of said naturally occurring polyA sequence of a third gene, wherein said second GT rich nucleic acid sequence comprises 0, 1, or 2 nucleotide modifications relative to the corresponding region of said naturally occurring polyA sequence of a third gene; and wherein said second GT rich nucleic acid region is positioned 5-100 nucleotides downstream of said first GT rich nucleic acid sequence.

In some embodiments, said third gene is a human gene. In some embodiments, said third gene is HGH. In some embodiments, said second GT rich nucleic acid sequence derived from said naturally occurring polyA sequence of a third gene comprises the nucleic acid sequence set forth in SEQ ID NO: 3. In some embodiments, said third gene is a non-human gene.

In some embodiments, said second gene and said third gene are different. In some embodiments, said second gene and said third gene are the same. In some embodiments, said second gene is HGH and said third gene is HGH.

In some embodiments, said polynucleotide comprises a sequence with at least 95%, 96%, 97%, 98%, 99%, or 100% identity to the sequence set forth in SEQ ID NO: 18.

In some embodiments, said polynucleotide comprises a nucleic acid sequence with 100% identity to the sequence set forth in SEQ ID NO: 18.

In some embodiments, said polynucleotide further comprises a first terminator positioned upstream or downstream of said polyA signal that comprises the nucleic acid sequence set forth in SEQ ID NO: 1.

In some embodiments, said first terminator is selected from the group consisting of a WPRE, a human C2 pause site element, a SV40 upstream sequence element, an alpha 2 globin pause site element, a human beta globin CoTC element, and a mouse beta-major globin pause site element.

In some embodiments, said first terminator comprises a human C2 gene pause site element, wherein said human C2 gene pause site element is positioned downstream of said polyA signal that comprises the nucleic acid sequence set forth in SEQ ID NO: 1, or a WPRE, wherein said WPRE is positioned upstream of said polyA signal that comprises the nucleic acid sequence set forth in SEQ ID NO: 1.

In some embodiments, said terminator comprises a nucleic acid sequence of at least 95%, 96%, 97%, 98%, 99%, or 100% identity to the sequence set forth in SEQ ID NO: 8 or 9.

In some embodiments, said polynucleotide comprises a second terminator.

In some embodiments, said first and said second terminator are different.

In some embodiments, said first terminator is a human C2 gene pause site element, wherein said first terminator is positioned downstream of said polyA signal that comprises the nucleic acid sequence set forth in SEQ ID NO: 1, and said second terminator is a WPRE, wherein said second terminator is positioned upstream of said polyA signal that comprises the nucleic acid sequence set forth in SEQ ID NO: 1.

In some embodiments, said first terminator comprises a nucleic acid sequence of at least 95%, 96%, 97%, 98%, 99%, or 100% identity to the sequence set forth in SEQ ID NO: 8; and said second terminator comprises a nucleic acid sequence of at least 95%, 96%, 97%, 98%, 99%, or 100% identity to the sequence set forth in SEQ ID NO: 9.

In some embodiments, upon inclusion in a suitable gene expression cassette, said polyA sequence mediates comparable or increased of a gene in said gene expression cassette relative to a control gene expression cassette that comprises a control polyA sequence.

In some embodiments, upon inclusion in a suitable gene expression cassette, said polyA sequence mediates at least a 2-fold, 3-fold, 4-fold, or 5-fold increase in expression of a gene in said gene expression relative to a control gene expression cassette that comprises a control polyA sequence.

In some embodiments, said polynucleotide does not contain a human miRNA binding site.

In some embodiments, said polynucleotide is a DNA polynucleotide. In one aspect provided herein are polynucleotides that are the complement of the polynucleotide described herein.

In one aspect, provided herein are RNA polynucleotides that are the RNA equivalent of the DNA polynucleotide described herein.

In one aspect, provided herein are polynucleotides comprising a terminator that comprises a nucleic acid sequence of at least 85%, 86%, 87%, 88%, 89%, 90%, 91%, 92%, 93%, 94%, 95%, 96%, 97%, 98%, 99%, or 100% identity to the sequence set forth in SEQ ID NO: 9.

In one aspect, provided herein are vectors comprising: a transgene that encodes a target protein; and a polynucleotide described herein. In some embodiments, said vector is a viral vector or a non-viral vector. In some embodiments, said vector is a non-viral vector and said non-viral vector is a plasmid. In some embodiments, said vector is a viral vector. In some embodiments, said viral vector is an adeno-associated virus (AAV) vector. In some embodiments, upon introduction into a host cell, said vector mediates comparable or increased expression of said gene relative to a control vector comprising a control polyA sequence. In some embodiments, upon introduction into a host cell, said vector mediates increased expression of said gene by at least 2-fold, 3-fold, 4-fold, or 5-fold relative to a control vector comprising a control polyA sequence.

In one aspect, provided herein are methods of expressing a transgene in a cell, said method comprising introducing a vector described herein into the cell.

In one aspect, provided herein is a method of modifying a cell, said method comprising introducing a polynucleotide described herein, or a vector described herein, into the cell.

In one aspect, provided herein is a cell comprising a polynucleotide described herein, or a vector described herein.

### BRIEF DESCRIPTION OF THE FIGURES

FIG. 1A is a schematic that shows the structure of the polyA sequence of a wild type human growth hormone (hGH) gene.

FIG. 1B is a schematic that shows a non-naturally occurring polyA sequence described further herein that comprises specific elements of a hGH polyA sequence, a SV40 late gene polyA sequence, and a rabbit beta globin (RBG) polyA sequence. The polyA sequence construct is referred to herein as SynHGH-V2 and is 135 bp in length.

FIG. 1C is a schematic that shows a non-naturally occurring polyA sequence described further herein that comprises specific elements from a SV40 late gene polyA sequence and a hGH polyA sequence. The polyA sequence construct is referred to herein as SynHGH-V3 and is 173 bp in length.

FIG. 2 is a dot graph that shows the expression of a luciferase reporter transgene expressed from the indicated vector and cell line (Huh7 or HepG2) normalized to a plasmid containing an SV40 polyA. The polyA sequence contained within the vector is indicated on the X axis (i.e., SV40, SynHGH-V2, or SynHGH-V3).

FIG. 3 is a dot graph that shows the expression of the luciferase reporter transgene expressed from the indicated vector and cell line (Huh7 or HepG2) normalized to a plasmid containing an SV40 upstream sequence element (USE) and an SV40 polyA. The polyA sequence contained within the vector is indicated on the X axis (i.e., SV40 USE+SV40, SynHGH-V2, or SynHGH-V3).

FIG. 4 is a dot graph that shows the average normalized expression of a luciferase reporter transgene expressed from the indicated vector and cell line (Huh7, HepG2, K562, HEK293, SVG p12, APRE-19). The polyA sequence contained within the vector is indicated on the X axis (i.e., SV40 (no terminator), SV40+Alpha 2 globin terminator, SV40+C2 terminator, SV40+human beta globin CoTC, SV40+mouse beta-major globin, or SV40+sWPRE terminator).

FIG. 5 is a dot graph that shows the average normalized expression of a luciferase reporter transgene expressed from the indicated vector and cell line (Huh7 or HepG2). The polyA sequence contained within the vector is indicated at the top of the graph (SV40, SynHGH-V2, or SynHGH-V3). The terminator is indicated on the X axis (i.e., WPRE, C2, or WPRE-C2).

### DETAILED DESCRIPTION

### Overview

The present disclosure provides, inter alia, non-naturally occurring polyA sequences that comprise a polyA signal and at least one GT rich region derived from a first naturally polyadenylation sequence (e.g., a polyadenylation sequence of a first gene), wherein either or both of i) the sequence immediately upstream of the polyadenylation signal, or ii) the sequence positioned between the polyadenylation signal and the at least one GT rich region, is replaced with a corresponding sequence derived from a second naturally occurring polyadenylation sequence (e.g., a polyadenylation sequence of a second gene), wherein said first and second polyadenylation sequences are different. In some embodiments, the first polyadenylation sequence is derived from a polyadenylation sequence of a first human gene and the second polyadenylation sequence is derived from a polyadenylation sequence from a second gene. In some embodiments, the first polyadenylation sequence is derived from a polyadenylation sequence of a human gene and the second polyadenylation sequence is derived from a polyadenylation sequence from a non-human gene (e.g., non-human mammal, virus, bacteria).

The non-naturally occurring polyA sequences described herein allow for optimization of polyA sequences such that expression of a transgene positioned 5′ (upstream) of the non-naturally occurring polyA sequence in a gene expression cassette is enhanced compared to the use of either of the natural occurring polyadenylation sequences (e.g., the first or second naturally occurring polyadenylation sequences). The non-naturally occurring polyA sequences described herein further allow for the use of specific elements from human polyadenylation sequences, while avoiding the potential for the human sequences to act as off-target homology arms in gene editing vectors for administration to humans.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting. The section headings used herein are for organizational purposes only and are not to be construed as limiting the subject matter described.

As used herein, the term “derived from” with reference to a nucleic acid sequence refers to a nucleic acid sequence that has at least 85% sequence identity to a reference naturally occurring nucleic acid sequence. For example, a GT rich region derived from a naturally occurring GT rich region of a human growth hormone means that the GT rich region has a nucleic acid sequence with at least 85% sequence identity to the sequence of the GT rich region of human growth hormone from which it is derived. The term “derived from” as used herein does not denote any specific process or method for obtaining the nucleic acid sequence. For example, the nucleic acid sequence can be chemically synthesized.

As used herein, the “polyA sequence” refers to a nucleic acid sequence that comprises from 5′ to 3′ a polyA signal (as defined herein) and a GT rich region (as defined herein), that can signal for the termination of transcription when placed 3′ of a coding sequence after the stop codon in a functional gene expression cassette that has any additional component necessary for expression of the coding sequence (e.g., a promoter).

As used herein, the term “polyA signal” refers to a six-nucleotide sequence located upstream of a GT rich region and facilitates polyadenylation. In some embodiments, the polyA signal comprises the well-known consensus (canonical) sequence set forth in SEQ ID NO: 1 (AATAAA), or a variant thereof that comprises the nucleic acid sequence of SEQ ID NO: 1 comprising 1 or 2 nucleotide modifications.

As used herein, the term “GT rich region” refers to a nucleic acid sequence that comprises at least 5 consecutive nucleobases of thymine (T) or guanine (G). For example, the exemplary nucleic acid sequences of GGGGG (SEQ ID NO: 29); TTTTT (SEQ ID NO: 30); GTGTG (SEQ ID NO: 31), would each meet the definition of “GT Rich Region” as used herein.

As used herein, the term “modification” with reference to a nucleic acid sequence as used herein refers a nucleic acid sequence that comprises at least one substitution, alteration, addition, or deletion of nucleotide compared to a reference nucleic acid sequence.

The terms “upstream sequence element” and “USE” are used interchangeably herein, and refer to a nucleic acid sequence located upstream of a polyA signal in a naturally occurring polyA sequence or derived from a naturally occurring polyA sequence.

The term “intervening sequence” with reference to a nucleic acid sequence as used herein, refers to a nucleic acid sequence that is positioned between (i.e., flanked) by two other defined sequences. For example, a nucleic acid sequence comprising from 5′ to 3′ a polyA signal sequence, an “X” nucleic acid sequence, and a GT rich region, the “X” nucleic acid sequence would qualify as an intervening sequence positioned between two other defined sequences (i.e., the polyA signal sequence and the GT rich region).

The term “terminator” with reference to a nucleic acid sequence as used herein refers to a nucleic acid sequence that directly or indirectly enhances posttranscriptional processing. Posttranscription processing includes, but is it not limited to, nuclear RNA processing, polyadenylation of RNA, nuclear export of RNA, and translation of RNA to protein. For example, a terminator may mediate release of the nascent RNA transcription from the RNA polymerase II complex; or the terminator my recruit one or more termination factor (e.g., a protein); or the terminator my enhance nuclear export of an RNA transcript, etc.

The term “identical” or “percent identity” with reference to a nucleic acid sequence or amino acid sequence refers to at least two nucleic acid or at least two amino acid sequences or subsequences that have a specified percentage of nucleotides or amino acids, respectively, that are the same, when compared and aligned for maximum correspondence, as measured using a sequence comparison algorithm or by visual inspection. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information.

### Non-Naturally Occurring Polyadenylation (PolyA) Sequences

In certain aspects, provided herein are non-naturally occurring polyA nucleic acid sequences. In some embodiments, the non-naturally occurring polyA sequence comprises from 5′ to 3′ a polyA signal and at least one GT rich region. In some embodiments, the non-naturally occurring polyA sequence further comprises an intervening sequence positioned between the polyA signal and the at least one GT rich region. In some embodiments, the non-naturally occurring polyA sequence comprises from 5′ to 3′ an upstream sequence, a polyA signal, and at least one GT rich region. In some embodiments, the non-naturally occurring polyA sequence comprises from 5′ to 3′ an upstream sequence, a polyA signal, an intervening sequence, and at least one GT rich region.

In some embodiments, the polyA sequence comprises a nucleic acid sequence derived from a polyA sequence of a first human gene and a nucleic acid sequence derived from a polyA sequence of a second human gene, wherein the first and second human genes are different.

In some embodiments, the non-naturally occurring polyA sequence comprises from 5′ to 3′ a polyA signal, an intervening sequence, and a GT rich region, wherein the polyA signal and the GT rich region are derived from a polyA sequence of a first human gene and the intervening sequence is derived from a polyA sequence of a second human gene. wherein the first and second human genes are different.

In some embodiments, the non-naturally occurring polyA sequence comprises from 5′ to 3′ a polyA signal, an intervening sequence, and a GT rich region, wherein the polyA signal and the GT rich region are derived from a polyA sequence of a first human gene and the intervening sequence is derived from a polyA sequence of a second human gene, wherein the first and second human genes are different.

In some embodiments, the non-naturally occurring polyA sequence comprises from 5′ to 3′ an upstream sequence element, a polyA signal, an intervening sequence, and a GT rich region, wherein the polyA signal, the GT rich region, and the intervening sequence are from a polyA sequence of a first human gene, and wherein the upstream sequence element is derived from a polyA sequence of a second human gene, wherein the first and second human genes are different.

In some embodiments, the polyA sequence comprises a nucleic acid sequence derived from a polyA sequence of a gene of one species (e.g., human gene) and a nucleic acid sequence derived from a polyA sequence of a gene from another species (e.g., a non-human gene).

In some embodiments, the non-naturally occurring polyA sequence comprises from 5′ to 3′ a polyA signal, an intervening sequence, and a GT rich region, wherein the polyA signal and the GT rich region are derived from a polyA sequence of a gene from one species (e.g., a human gene) and the intervening nucleic acid sequence is derived from a polyA sequence of a gene from another species (e.g., a non-human gene).

In some embodiments, the non-naturally occurring polyA sequence comprises from 5′ to 3′ a polyA signal, an intervening sequence, and a GT rich region, wherein the polyA signal and the GT rich region are derived from a polyA sequence of a human gene the intervening sequence is derived from a polyA sequence of a non-human gene.

In some embodiments, the non-naturally occurring polyA sequence comprises from 5′ to 3′ an upstream sequence element, a polyA signal, an intervening sequence, and a GT rich region, wherein the polyA signal, the GT rich region, and the intervening sequence are from a polyA sequence of a gene from one species (e.g., a human gene), and wherein the upstream sequence element is from a polyA sequence of a gene from another species (e.g., a non-human gene).

In some embodiments, the non-naturally occurring polyA sequence comprises from 5′ to 3′ an upstream sequence element, a polyA signal, an intervening sequence, and a GT rich region, wherein the polyA signal, the GT rich region, and the intervening sequence are from a polyA sequence of a human gene, and wherein the upstream sequence element is derived from a polyA sequence of a non-human gene. In some embodiments, the human gene is selected from the group consisting of human growth hormone or human albumin. In some embodiments, the non-human gene is a viral, bacterial, or non-human mammal gene. In some embodiments, the non-human gene is a viral gene. In some embodiments, the viral gene is simian virus 40 (SV40) late gene, herpes simplex virus, or Autographa californica nuclear polyhedrosis virus. In some embodiments, the non-human gene is a non-human mammalian gene. In some embodiments, the non-human mammalian gene is a rabbit gene, cow gene, mouse gene, rat gene, or hamster gene. In some embodiments, the non-human mammalian gene is rabbit beta globin. In some embodiments, the non-human gene is bovine growth hormone.

In some embodiments, the polyA sequence comprises a nucleic acid sequence derived from a naturally occurring polyA sequence of a human gene and a nucleic acid sequence derived from a naturally occurring polyA sequence derived from a non-human gene. In some embodiments, the polyA sequence comprises a nucleic acid sequence wherein no more than 10%, 20%, 30%, 40%, 50%, 60%, 70%, 80%, or 90% of the nucleic acid sequence is derived from a human polyA sequence. In some embodiments, the polyA sequence comprises a nucleic acid sequence wherein less than 10%, 20%, 30%, 40%, 50%, 60%, 70%, 80%, or 90% of the nucleic acid sequence is derived from a human polyA sequence. In some embodiments, the polyA sequence comprises a nucleic acid sequence wherein from about 10%-90%, 10%-80%, 10%-70%, 10%-60%, 10%-50%, 10%-40%, 10%-30%, 10%-20%, 20%-90%, 30%-90%, 40%-90%, 50%-90%, 60%-90%, or 70%-90% of the nucleic acid sequence is derived from a human polyA sequence.

In some embodiments, the polyA sequence is no more than 500, 450, 400, 350, 300, 350, or 200 nucleotides in length. In some embodiments, the polyA sequence is at least 100, 200, 300, 400, or 500 nucleotides in length. In some embodiments, the polyA sequence is from about 200-600, 250-600, 300-600, 350-600, 400-600, 450-600, 500-600, 550-600, 200-500, 250-500, 300-500, 350-500, 400-500, 450-500, 300-500, 350-500, 400-500, or 450-500 nucleotides in length.

### PolyA Signal

In some embodiments, a non-naturally occurring polyA sequence described herein comprises a polyA signal. In some embodiments, the polyA signal is derived from a naturally occurring polyA sequence. In some embodiments, the polyA signal is derived from a naturally occurring polyA sequence, and comprises 1, 2, or 3 nucleotide modifications relative to the naturally occurring polyA sequence form which it is derived. In some embodiments, the polyA signal is a variant of the consensus sequence of SEQ ID NO: 1 (AATAAA). In some embodiments, the polyA signal comprises the nucleic acid sequence of SEQ ID NO: 1 (AATAAA), with 1, 2, or 3 nucleotide modifications. In some embodiments, the polyA signal comprises the consensus nucleic acid sequence as set forth in SEQ ID NO: 1 (AATAAA). In some embodiments, the polyA signal consists essentially of the consensus nucleic acid sequence as set forth in SEQ ID NO: 1 (AATAAA). In some embodiments, the polyA signal consists of the consensus nucleic acid sequence as set forth in SEQ ID NO: 1 (AATAAA).

In some embodiments, the polyA signal comprises a non-consensus polyA signal. Exemplary non-consensus polyA signals are provided in Table 1. In some embodiments, the polyA signal sequence comprises the nucleic acid sequence of SEQ ID NO: 32 (ATTAAA). In some embodiments, the polyA signal sequence comprises the nucleic acid sequence of SEQ ID NO: 33 (AGTAAA). In some embodiments, the polyA signal sequence comprises the nucleic acid sequence of SEQ ID NO: 34 (TATAAA). In some embodiments, the polyA signal sequence comprises the nucleic acid sequence of SEQ ID NO: 35 (CATAAA). In some embodiments, the polyA signal sequence comprises the nucleic acid sequence of SEQ ID NO: 36 (GATAAA). In some embodiments, the polyA signal sequence comprises the nucleic acid sequence of SEQ ID NO: 37 (AATATA). In some embodiments, the polyA signal sequence comprises the nucleic acid sequence of SEQ ID NO: 38 (AATACA). In some embodiments, the polyA signal sequence comprises the nucleic acid sequence of SEQ ID NO: 39 (AATAGA). In some embodiments, the polyA signal sequence comprises the nucleic acid sequence of SEQ ID NO: 40 (ACTAAA). In some embodiments, the polyA signal sequence comprises the nucleic acid sequence of SEQ ID NO: 41 (AAGAAA). In some embodiments, the polyA signal sequence comprises the nucleic acid sequence of SEQ ID NO: 42 (AATGAA).

TABLE 1
Exemplary PolyA Signal Sequences

| | Nucleic | SEQ |
|---|---|---|
| | Acid | ID |
| Name | Sequence | NO |
| Consensus | AATAAA | 1 |
| Non-consensus -1 | ATTAAA | 32 |
| Non-consensus -2 | AGTAAA | 33 |
| Non-consensus -3 | TATAAA | 34 |
| Non-consensus -4 | CATAAA | 35 |
| Non-consensus -5 | GATAAA | 36 |
| Non-consensus -6 | AATATA | 37 |
| Non-consensus -7 | AATACA | 38 |
| Non-consensus -8 | AATAGA | 39 |
| Non-consensus -9 | AGTAAA | 40 |
| Non-consensus -10 | AAGAAA | 41 |
| Non-consensus -11 | AATGAA | 42 |

In some embodiments, the polyA sequences comprises a polyA signal and a GT rich region. In some embodiments, the polyA signal is positioned from about 10-40, 10-30, 10-20, 15-40, 15-30, or 15-20 nucleotides upstream (5′) of the GT rich region in a non-naturally occurring polyA sequence described herein. In some embodiments, the polyA signal is positioned from about 10-30 nucleotides upstream (5′) of the GT rich region in non-naturally occurring polyA sequence described herein. In some embodiments, the polyA signal is positioned from about 15-30 nucleotides upstream (5′) of the GT rich region in non-naturally occurring polyA sequence described herein. In some embodiments, the polyA signal is positioned from about 15-25 nucleotides upstream (5′) of the GT rich region in non-naturally occurring polyA sequence described herein. In some embodiments, the polyA signal is positioned from about 15-20 nucleotides upstream (5′) of the GT rich region in non-naturally occurring polyA sequence described herein. In some embodiments, the polyA signal is positioned from about 15, 16, 17, 18, 19, 20, 21, 22, 23, 24, 25, 26, 27, 28, 29, or 30 nucleotides upstream (5′) of the GT rich region in non-naturally occurring polyA sequence described herein. In some embodiments, the polyA signal is positioned from about 19, 20, 21, or 22 nucleotides upstream (5′) of the polyA signal in non-naturally occurring polyA sequence described herein.

### GT Rich Region

In some embodiments, a non-naturally occurring polyA sequence described herein comprises a GT rich region. In some embodiments, the GT rich region is derived from a GT rich region of a naturally occurring polyA sequence. In some embodiments, the GT rich region comprises at least 95%, 96%, 97%, 98%, 99%, or 100% sequence identity to the GT rich region of the naturally occurring polyA sequence from which it is derived. In some embodiments, the GT rich region comprises 1, 2, 3, 4, or 5 nucleotide modifications compared to the naturally occurring polyA sequence from which it is derived. In some embodiments, the GT rich region comprises a nucleotide modification at the 3′ or 5′ end of the nucleotide sequence, compared to the naturally occurring polyA sequence from which it is derived. In some embodiments, the GT rich region is derived from a GT rich region of a naturally occurring polyA sequence of a human gene. In some embodiments, the GT rich region is derived from a GT rich region of a naturally occurring polyA sequence of a non-human gene.

In some embodiments, the GT rich region is derived from human growth hormone (HGH) gene. In some embodiments, the GT rich region is derived from rabbit beta-globin. In some embodiments, the GT rich region comprises the nucleic acid sequence set forth in SEQ ID NO: 2, with 1, 2, or 3, nucleotide modifications compared to the nucleic acid sequence set forth in SEQ ID NO: 2. In some embodiments, the GT rich region comprises the nucleic acid sequence set forth in SEQ ID NO: 2. In some embodiments, the GT rich region consists essentially of the nucleic acid sequence set forth in SEQ ID NO: 2. In some embodiments, the GT rich region consists of the nucleic acid sequence set forth in SEQ ID NO: 2.

In some embodiments, the GT rich region comprises the nucleic acid sequence set forth in SEQ ID NO: 3, with 1, 2, 3, 4, or 5 nucleotide modifications compared to the nucleic acid sequence set forth in SEQ ID NO: 3. In some embodiments, the GT rich region comprises the nucleic acid sequence set forth in SEQ ID NO: 3. In some embodiments, the GT rich region consists essentially of the nucleic acid sequence set forth in SEQ ID NO: 3. In some embodiments, the GT rich region consists of the nucleic acid sequence set forth in SEQ ID NO: 3.

In some embodiments, a polyA sequence comprises a GT rich region and a polyA signal. In some embodiments, the GT rich region is positioned from about 10-40, 10-30, 10-20, 15-40, 15-30, or 15-20 nucleotides downstream (3′) of the polyA signal. In some embodiments, the GT rich region is positioned from about 10-30 nucleotides downstream (3′) of the polyA signal. In some embodiments, the GT rich region is positioned from about 15-30 nucleotides downstream (3′) of the polyA signal. In some embodiments, the GT rich region is positioned from about 15-25 nucleotides downstream (3′) of the polyA signal. In some embodiments, the GT rich region is positioned from about 15-20 nucleotides downstream (3′) of the polyA signal. In some embodiments, the GT rich region is positioned from about 15, 16, 17, 18, 19, 20, 21, 22, 23, 24, 25, 26, 27, 28, 29, or 30 nucleotides downstream (3′) of the polyA signal. In some embodiments, the GT rich region is positioned from about 19, 20, 21, or 22 nucleotides downstream (3′) of the polyA signal.

In some embodiments, the GT rich region comprises a nucleic acid sequence of at least 3, 4, 5, 6, 7, 8, 9, 10, 11, 12, 13, 14, 15, 16, 17, 18, 19, or 20 nucleotides. In some embodiments, the GT rich region comprises a nucleic acid sequence of no more than 3, 4, 5, 6, 7, 8, 9, 10, 11, 12, 13, 14, 15, 16, 17, 18, 19, 20, 30, 40, or 50 nucleotides. In some embodiments, the GT rich region comprises a nucleic acid sequence from about 3-20, 3-19, 3-18, 3-17, 3-16, 3-15, 3-14, 3-13, 3-12, 3-11, 3-10, 3-9, 3-8, 3-7, 3-6, 3-5, 3-4, 5-20, 5-19, 5-18, 5-17, 5-16, 5-15, 5-14, 5-13, 5-14, 5-13, 5-12, 5-11, 5-10, 5-9, 5-8, 5-7, or 5-6 nucleotides.

In some embodiments, a non-naturally occurring polyA sequence described herein comprises at least 2 GT rich regions (a first GT rich region and a second GT rich region). In some embodiments, the first GT rich region and a second GT rich region are both derived from a naturally occurring polyA sequence. In some embodiments, the first GT rich region and a second GT rich region both derived from the same naturally occurring polyA sequence. In some embodiments, the first GT rich region and a second GT rich region are derived from different naturally occurring polyA sequences. In some embodiments, the first and/or second of said two GT rich regions comprises 1, 2, 3, 4, or 5 nucleotide modifications compared to the naturally occurring polyA sequence from which each is derived.

In some embodiments, the first GT rich region is derived from a GT rich region of a naturally occurring polyA sequence of a human gene. In some embodiments, the first GT rich region is derived from a GT rich region of a naturally occurring polyA sequence of a non-human gene.

In some embodiments, the first GT rich region comprises 1, 2, 3, 4, or 5 nucleotide modifications compared to the naturally occurring polyA sequence from which it is derived. In some embodiments, the first GT rich region comprises a nucleotide modification at the 3′ or 5′ end of the nucleotide sequence, compared to the naturally occurring polyA sequence from which it is derived.

In some embodiments, the first GT rich region comprises the nucleic acid sequence set forth in SEQ ID NO: 2, with 1, 2, or 3 nucleotide modifications compared to the nucleic acid sequence set forth in SEQ ID NO: 2. In some embodiments, the first GT rich region comprises the nucleic acid sequence set forth in SEQ ID NO: 2.

In some embodiments, the second GT rich region comprises 1, 2, 3, 4, or 5 nucleotide modifications compared to the naturally occurring polyA sequence from which it is derived. In some embodiments, the second GT rich region comprises a nucleotide modification at the 3′ or 5′ end of the nucleotide sequence, compared to the naturally occurring polyA sequence from which it is derived.

In some embodiments, the second GT rich region is derived from a GT rich region of a naturally occurring polyA sequence of a human gene. In some embodiments, the second GT rich region is derived from a GT rich region of a naturally occurring polyA sequence of a non-human gene.

In some embodiments, the second GT rich region comprises the nucleic acid sequence set forth in SEQ ID NO: 3, with 1, 2, 3, 4, or 5 nucleotide modifications compared to the nucleic acid sequence set forth in SEQ ID NO: 3. In some embodiments, the second GT rich region comprises the nucleic acid sequence set forth in SEQ ID NO: 3.

In some embodiments, the first GT rich region comprises a nucleic acid sequence of at least 3, 4, 5, 6, 7, 8, 9, 10, 11, 12, 13, 14, 15, 16, 17, 18, 19, or 20 nucleotides. In some embodiments, the GT rich region comprises a nucleic acid sequence of no more than 3, 4, 5, 6, 7, 8, 9, 10, 11, 12, 13, 14, 15, 16, 17, 18, 19, 20, 30, 40, or 50 nucleotides. In some embodiments, the first GT rich region comprises a nucleic acid sequence from about 3-20, 3-19, 3-18, 3-17, 3-16, 3-15, 3-14, 3-13, 3-12, 3-11, 3-10, 3-9, 3-8, 3-7, 3-6, 3-5, 3-4, 5-20, 5-19, 5-18, 5-17, 5-16, 5-15, 5-14, 5-13, 5-14, 5-13, 5-12, 5-11, 5-10, 5-9, 5-8, 5-7, or 5-6 nucleotides.

In some embodiments, the second GT rich region comprises a nucleic acid sequence of at least 3, 4, 5, 6, 7, 8, 9, 10, 11, 12, 13, 14, 15, 16, 17, 18, 19, or 20 nucleotides. In some embodiments, the GT rich region comprises a nucleic acid sequence of no more than 3, 4, 5, 6, 7, 8, 9, 10, 11, 12, 13, 14, 15, 16, 17, 18, 19, 20, 30, 40, or 50 nucleotides. In some embodiments, the second GT rich region comprises a nucleic acid sequence from about 3-20, 3-19, 3-18, 3-17, 3-16, 3-15, 3-14, 3-13, 3-12, 3-11, 3-10, 3-9, 3-8, 3-7, 3-6, 3-5, 3-4, 5-20, 5-19, 5-18, 5-17, 5-16, 5-15, 5-14, 5-13, 5-14, 5-13, 5-12, 5-11, 5-10, 5-9, 5-8, 5-7, or 5-6 nucleotides.

In some embodiments, the first GT rich region is located upstream (5′) of the second GT rich region. In some embodiments, the first GT rich region is positioned from about 15-20 nucleotides downstream (3′) of a polyA signal in non-naturally occurring polyA sequence described herein. In some embodiments, the first GT rich region is positioned from about 15, 16, 17, 18, 19, 20, 21, 22, 23, 24, 25, 26, 27, 28, 29, or 30 nucleotides downstream (3′) of a polyA signal in non-naturally occurring polyA sequence described herein. In some embodiments, the first GT rich region is positioned from about 19, 20, 21, or 22 nucleotides downstream (3′) of a polyA signal in non-naturally occurring polyA sequence described herein.

In some embodiments, the second GT rich region is located downstream (3′) of the first GT rich region. In some embodiments, the second GT rich region is positioned from about 1-100, 1-50, 1-25, 1-20, 1-15, 1-10, 1-5, 5-100, 5-50, 5-25, 5-20, 5-15, 5-10, 10-100, 10-50, 10-25, or 10-20 nucleotides downstream (3′) of the first GT rich region. In some embodiments, the second GT rich region is positioned from about 100, 90, 80, 70, 60, 50, 40, 30, 20, 19, 18, 17, 16, 15, 14, 13, 12, 11, 10, 9, 8, 7, 6, 5, 4, 3, 2, 1, or 0 (no intervening nucleotides) nucleotides downstream (3′) of the first GT rich region.

In some embodiments, wherein the first GT rich region and the second GT rich region are derived from the same naturally occurring polyA sequence, the spacing between the first GT rich region and the second GT rich region (i.e., the number of nucleotides positioned between the first GT rich region and the second GT rich region) is the same as in the naturally occurring polyA sequence. In some embodiments, wherein the first GT rich region and the second GT rich region are derived from the same naturally occurring polyA sequence, the spacing between the first GT rich region and the second GT rich region (i.e., the number of nucleotides positioned between the first GT rich region and the second GT rich region) is the same as in the naturally occurring polyA sequence—plus or minus up to 1, 2, 3, 4, or 5 nucleotides.

The nucleic acid sequences of exemplary GT rich regions are provided in Table 2.

TABLE 2
Exemplary GT Rich Regions

| | | SEQ |
|---|---|---|
| | | ID |
| Name | Nucleic Acid Sequence | NO |
| T rich | TTTTGTCT | 2 |
| region | | |
| G rich | GGGGTGGAGGGGGGTGGTATGGAGCAAGGGG | 3 |
| region | | |

### Intervening Sequences

In some embodiments, a non-naturally occurring polyA sequence described herein comprises an intervening nucleic acid sequence. In some embodiments, the intervening nucleic acid sequence is derived from a naturally occurring polyA sequence. In some embodiments, the intervening nucleic acid sequence is derived from a naturally occurring polyA sequence of a human gene. In some embodiments, the intervening nucleic acid sequence is derived from a naturally occurring polyA sequence of a non-human gene. In some embodiments, the intervening sequence mediates a specific function, e.g., enhances efficiency of transcription termination compared to a comparable control polyadenylation sequence comprising a control intervening sequence (e.g., naturally occurring).

In some embodiments the intervening sequence comprises at least 5, 6, 7, 8, 9, 10, 11, 12, 13, 14, 15, 20, 25, 30, 35, 40, 50, or 100 nucleotides. In some embodiments the intervening sequence comprises no more than 5, 6, 7, 8, 9, 10, 11, 12, 13, 14, 15, 20, 25, 30, 35, 40, 50, 100, 150, or 200 nucleotides. In some embodiments the intervening sequence comprises from about 5-100, 5-50, 5-25, 5-10, 10-100, 10-50, 10-40, 10-30, or 10-20 nucleotides.

In some embodiments, the intervening sequence is derived from a naturally occurring polyA sequence and comprises at least a portion of the nucleic acid sequence positioned between a polyA signal and a GT rich region. In some embodiments, the intervening sequence is derived from a naturally occurring polyA sequence and comprises the nucleic acid sequence positioned between a polyA signal and a GT rich region, with 1, 2, 3, 4, 5, 6, 7, 8, 9, or 10 nucleotide modifications, additions, or deletions on the 3′ and/or 5′ end of the naturally occurring intervening sequence. In some embodiments, the intervening sequence is derived from a naturally occurring polyA sequence and comprises a nucleic acid sequence that has at least 90%, 91%, 92%, 93%, 93%, 94%, 95%, 96%, 97%, 98%, 99%, or 100% identity to the nucleic acid sequence positioned between a polyA signal and a GT rich region of the naturally occurring polyA sequence.

In some embodiments, the intervening sequence is derived from a viral gene. In some embodiments, the intervening sequence is derived from a simian virus 40 (SV40) late gene. In some embodiments, the intervening sequence comprises the nucleic acid sequence set forth in SEQ ID NO: 4, with 1, 2, 3, 4, or 5 nucleotide modifications compared to the nucleic acid sequence set forth in SEQ ID NO: 4. In some embodiments, the intervening sequence comprises the nucleic acid sequence set forth in SEQ ID NO: 4.

In some embodiments, the intervening sequence is derived from a naturally occurring polyA sequence and comprises at least a portion of the nucleic acid sequence positioned between a first GT rich region and a second GT rich region of the naturally occurring polyA sequence. In some embodiments, the intervening sequence is derived from a naturally occurring polyA sequence and comprises the nucleic acid sequence positioned between a first GT rich region and a second GT rich region, with 0, 1, 2, 3, 4, 5, 6, 7, 8, 9, or 10 nucleotide modifications, additions, or deletions on the 3′ and/or 5′ end of the naturally occurring intervening sequence. In some embodiments, the intervening sequence is derived from a naturally occurring polyA sequence and comprises a nucleic acid sequence that has at least 90%, 91%, 92%, 93%, 93%, 94%, 95%, 96%, 97%, 98%, 99%, or 100% identity to the nucleic acid sequence positioned between a first GT rich region and a second GT rich region of the naturally occurring polyA sequence.

In some embodiments, the intervening sequence is derived from a non-human mammal gene. In some embodiments, the intervening sequence is derived from a non-human mammal gene is bovine growth hormone (BGH) or rabbit beta globin (RBG). In some embodiments, the intervening sequence comprises the nucleic acid sequence set forth in SEQ ID NO: 5, with 1, 2, 3, 4, or 5 nucleotide modifications compared to the nucleic acid sequence set forth in SEQ ID NO: 5. In some embodiments, the intervening sequence comprises the nucleic acid sequence set forth in SEQ ID NO: 5.

In some embodiments, the polyA sequence comprises multiple (i.e., 2 or more) intervening sequences. In some embodiments, the multiple intervening sequences are derived from the same naturally occurring polyA sequence. In some embodiments, the multiple intervening sequences are derived from different naturally occurring polyA sequences. In some embodiments, the multiple intervening sequences are derived from different naturally occurring polyA sequences from different species.

In some embodiments, the polyA sequence comprises a first intervening sequence and a second intervening sequence. In some embodiments, the first and second intervening sequences are different. In some embodiments, the first intervening sequence and the second intervening sequence are derived from the same naturally occurring polyA sequence. In some embodiments, the first intervening sequence and the second intervening sequence are derived from different naturally occurring polyA sequences. In some embodiments, the naturally occurring polyA sequence is a naturally occurring polyA sequence of a non-human gene. In some embodiments, the naturally occurring polyA sequence is a naturally occurring polyA sequence of a human gene.

In some embodiments, the first intervening sequence is derived from a naturally occurring polyA sequence and comprises at least a portion of the nucleic acid sequence positioned between a polyA signal and a GT rich region. In some embodiments, the intervening sequence is derived from a naturally occurring polyA sequence and comprises the nucleic acid sequence positioned between a polyA signal and a GT rich region, with 1, 2, 3, 4, 5, 6, 7, 8, 9, or 10 nucleotide modifications, additions, or deletions on the 3′ and/or 5′ end of the naturally occurring intervening sequence. In some embodiments, the intervening sequence is derived from a naturally occurring polyA sequence and comprises a nucleic acid sequence that has at least 90%, 91%, 92%, 93%, 93%, 94%, 95%, 96%, 97%, 98%, 99%, or 100% identity to the nucleic acid sequence positioned between a polyA signal and a GT rich region of the naturally occurring polyA sequence.

In some embodiments, the second intervening sequence is derived from a naturally occurring polyA sequence and comprises at least a portion of the nucleic acid sequence positioned between a first GT rich region and a second GT rich region of the naturally occurring polyA sequence. In some embodiments, the intervening sequence is derived from a naturally occurring polyA sequence and comprises the nucleic acid sequence positioned between a first GT rich region and a second GT rich region, with 1, 2, 3, 4, 5, 6, 7, 8, 9, or 10 nucleotide modifications, additions, or deletions on the 3′ and/or 5′ end of the naturally occurring intervening sequence. In some embodiments, the intervening sequence is derived from a naturally occurring polyA sequence and comprises a nucleic acid sequence that has at least 90%, 91%, 92%, 93%, 93%, 94%, 95%, 96%, 97%, 98%, 99%, or 100% identity to the nucleic acid sequence positioned between a first GT rich region and a second GT rich region of the naturally occurring polyA sequence.

In some embodiments, the intervening sequence is derived from a viral gene. In some embodiments, is derived from a simian virus 40 (SV40) late gene. In some embodiments, the first intervening sequence comprises the nucleic acid sequence set forth in SEQ ID NO: 4, with 1, 2, 3, 4, or 5 nucleotide modifications compared to the nucleic acid sequence set forth in SEQ ID NO: 4. In some embodiments, the first intervening sequence comprises the nucleic acid sequence set forth in SEQ ID NO: 4.

In some embodiments, the intervening sequence is derived from a non-human mammal gene. In some embodiments, is derived from a non-human mammal gene is bovine growth hormone (BGH) or rabbit beta globin (RBG). In some embodiments, the second intervening sequence comprises the nucleic acid sequence set forth in SEQ ID NO: 5, with 1, 2, 3, 4, or 5 nucleotide modifications compared to the nucleic acid sequence set forth in SEQ ID NO: 5. In some embodiments, the second intervening sequence comprises the nucleic acid sequence set forth in SEQ ID NO: 5.

The nucleic acid sequences of exemplary intervening sequences are provided in Table 3.

TABLE 3
Exemplary Intervening Sequences

| | | SEQ |
|---|---|---|
| | | ID |
| Name | Nucleic Acid Sequence | NO |
| SV40 | CAAGTTAACAACAA | 4 |
| sequence | | |
| RBG | CGTGTGTTGGAATTTTTTGTGTCTCT | 5 |
| region | | |

### Upstream Sequence Elements (USE)

In some embodiments, a non-naturally occurring polyA sequence described herein comprises an upstream sequence element. In some embodiments, the upstream sequence element is derived from a naturally occurring polyA sequence. In some embodiments, the upstream sequence element is derived from a naturally occurring polyA sequence of a human gene. In some embodiments, the upstream sequence element is derived from a naturally occurring polyA sequence of a human gene.

In some embodiments, the upstream sequence element comprises at least 95%, 96%, 97%, 98%, 99%, or 100% sequence identity to the upstream sequence element of the naturally occurring polyA sequence from which it is derived. In some embodiments, the upstream sequence element comprises 1, 2, 3, 4, or 5 nucleotide modifications compared to the naturally occurring polyA sequence from which it is derived. In some embodiments, the upstream sequence element comprises a nucleotide modification at the 3′ or 5′ end of the nucleotide sequence, compared to the naturally occurring polyA sequence from which it is derived.

In some embodiments the upstream sequence element comprises at least 10, 11, 12, 13, 14, 15, 20, 25, 30, 35, 40, 50, 60, 70, 80, 90, or 100 nucleotides. In some embodiments the upstream sequence element comprises no more than 5, 6, 7, 8, 9, 10, 11, 12, 13, 14, 15, 20, 25, 30, 35, 40, 50, or 100 nucleotides. In some embodiments the upstream sequence element comprises from about 5-200, 5-100, 5-50, 5-25, 10-200, 10-100, 10-50, 10-25, 50-200, 50-100, or 50-75.

In some embodiments, the upstream sequence element comprises at least 1, 2, 3, 4, or 5 repeats of a single nucleic acid sequence derived from a naturally occurring polyA sequence. In some embodiments, the upstream sequence element comprises at least 1, 2, 3, 4, or 5 repeats of a single nucleic acid sequence derived from a naturally occurring polyA sequence.

In some embodiments, the upstream sequence element is derived from a polyA sequence of a viral gene. In some embodiments, the viral gene is simian virus 40 (SV40) late gene. In some embodiments, the upstream sequence element comprises the nucleic acid sequence set forth in SEQ ID NO: 13, with 1, 2, 3, 4, or 5 nucleotide modifications compared to the nucleic acid sequence set forth in SEQ ID NO: 13. In some embodiments, the upstream sequence element comprises the nucleic acid sequence set forth in SEQ ID NO: 13. In some embodiments, the upstream sequence element consists essentially of the nucleic acid sequence set forth in SEQ ID NO: 13. In some embodiments, the upstream sequence element consists of the nucleic acid sequence set forth in SEQ ID NO: 13.

In some embodiments, the upstream sequence element comprises the nucleic acid sequence set forth in SEQ ID NO: 14, with 1, 2, 3, 4, or 5 nucleotide modifications compared to the nucleic acid sequence set forth in SEQ ID NO: 14. In some embodiments, the upstream sequence element comprises the nucleic acid sequence set forth in SEQ ID NO: 14. In some embodiments, the upstream sequence element consists essentially of the nucleic acid sequence set forth in SEQ ID NO: 14. In some embodiments, the upstream sequence element consists of the nucleic acid sequence set forth in SEQ ID NO: 14.

In some embodiments, the upstream sequence element comprises the nucleic acid sequence set forth in SEQ ID NO: 15, with 1, 2, 3, 4, or 5 nucleotide modifications compared to the nucleic acid sequence set forth in SEQ ID NO: 15. In some embodiments, the upstream sequence element comprises the nucleic acid sequence set forth in SEQ ID NO: 15. In some embodiments, the upstream sequence element consists essentially of the nucleic acid sequence set forth in SEQ ID NO: 15. In some embodiments, the upstream sequence element consists of the nucleic acid sequence set forth in SEQ ID NO: 15.

In some embodiments, the upstream sequence element comprises the nucleic acid sequence set forth in SEQ ID NO: 16, with 1, 2, 3, 4, or 5 nucleotide modifications compared to the nucleic acid sequence set forth in SEQ ID NO: 16. In some embodiments, the upstream sequence element comprises the nucleic acid sequence set forth in SEQ ID NO: 16. In some embodiments, the upstream sequence element consists essentially of the nucleic acid sequence set forth in SEQ ID NO: 16. In some embodiments, the upstream sequence element consists of the nucleic acid sequence set forth in SEQ ID NO: 16.

The nucleic acid sequence of exemplary upstream sequence elements is provided in Table 4.

TABLE 4
Exemplary Upstream Sequence Elements

| | | SEQ |
|---|---|---|
| | | ID |
| Name | Nucleic Acid Sequence | NO |
| SV40 1X | TTTATTTGTGAAATTTGTGATGCTATTGCT | 13 |
| (with 3′ T | TTATTTGTAACCAC | |
| to C modi- | | |
| fication) | | |
| SV40 1X | TTTATTTGTGAAATTTGTGATGCTATTGCT | 14 |
| (without | TTATTTGTAACCAT | |
| 3′ T to C | | |
| modifica- | | |
| tion) | | |
| SV40 2X | TTTATTTGTGAAATTTGTGATGCTATTGCT | 15 |
| (with 3′ T | TTATTTGTAACCATTTTATTTGTGAAATTT | |
| to C modi- | GTGATGCTATTGCTTTATTTGTAACCAC | |
| fication) | | |
| SV40 2X | TTTATTTGTGAAATTTGTGATGCTATTGCT | 16 |
| (without | TTATTTGTAACCATTTTATTTGTGAAATTT | |
| 3′ T to C | GTGATGCTATTGCTTTATTTGTAACCAT | |
| modifica- | | |
| tion) | | |

### Terminators

In some embodiments, a non-naturally occurring polyA sequence described herein comprises a terminator. In some embodiments, the terminator that is derived from a naturally occurring polyA sequence. In some embodiments, the terminator is derived from a naturally occurring polyA sequence of a human gene. In some embodiments, the terminator is derived from a naturally occurring polyA sequence of a non-human gene. In some embodiments, the terminator is not derived from a naturally occurring polyA sequence of a non-human gene. In some embodiments, the terminator is derived from a naturally occurring terminator sequence that is 3′ (downstream) of a gene's 3′ UTR.

In some embodiments, the non-naturally occurring polyA sequence comprises a polyA signal, a GT rich region, and a terminator. In some embodiments, the terminator is positioned 3′ (downstream) of said polyA signal and said GT rich region. In some embodiments, the terminator is positioned 5′ (upstream) of said polyA signal. In some embodiments, the terminator is positioned 5′ (upstream) of said polyA signal and said GT rich region.

In some embodiments, the terminator is a human C2 pause site. In some embodiments, the terminator is a SV40 upstream sequence element (USE). In some embodiments, the terminator is an alpha 2 globin pause site. In some embodiments, the terminator is a human beta globin CoTC. In some embodiments, the terminator is a mouse beta-major globin pause site. In some embodiments, the terminator is a woodchuck hepatitis virus posttranscriptional regulatory element (WPRE) from strain woodchuck hepatitis virus (WHV) strain (GenBank: 702442.1) (SEQ IS NO: 53). In some embodiments, the terminator is a WPRE from WHV strain WHV8 (GenBank: J04514.1) (SEQ ID NO: 52).

Exemplary terminators include woodchuck hepatitis virus posttranscriptional regulatory elements (WPRE). In some embodiments, the terminator is a WPRE. In some embodiments, the WPRE sequence is modified (e.g., to improve the safety profile of the WPRE). Exemplary modifications include those described by in Schambach A et al., Woodchuck hepatitis virus post-transcriptional regulatory element deleted from X protein and promoter sequences enhances retroviral vector titer and expression, Gene Ther. 2006; 13(7): 641-645. doi:10.1038/sj.gt.3302698 (the contents of which are incorporated by reference herein). Exemplary modifications include, but are not limited to, removal of the protein X promoter and coding sequence, and mutation of all relevant “ATG”s to “TGG” or “CGG.”

In some embodiments, the terminator is a WPRE comprising the nucleic acid sequence of SEQ ID NO: 9. In some embodiments, the terminator comprises the nucleic acid sequence set forth in SEQ ID NO: 9, with 1, 2, 3, 4, or 5 nucleotide modifications compared to the nucleic acid sequence set forth in SEQ ID NO: 9. In some embodiments, the terminator comprises the nucleic acid sequence set forth in SEQ ID NO: 9, with 1, 2, 3, 4, or 5 nucleotide deletions compared to the nucleic acid sequence set forth in SEQ ID NO: 9. In some embodiments, the terminator comprises a nucleic acid sequence at least 85%, 86%, 87%, 88%, 89%, 90%, 91%, 92%, 93%, 94%, 95%, 96%, 97%, 98%, 99%, or 100% identical to the nucleic acid sequence set forth in SEQ ID NO: 9. In some embodiments, the terminator comprises the nucleic acid sequence set forth in SEQ ID NO: 9. In some embodiments, the terminator consists essentially of the nucleic acid sequence set forth in SEQ ID NO: 9. In some embodiments, the terminator consists of the nucleic acid sequence set forth in SEQ ID NO: 9.

In some embodiments, the terminator is a WPRE comprising the nucleic acid sequence of SEQ ID NO: 52. In some embodiments, the terminator comprises the nucleic acid sequence set forth in SEQ ID NO: 52, with 1, 2, 3, 4, 5, 10, 15, or 20 nucleotide modifications compared to the nucleic acid sequence set forth in SEQ ID NO: 52. In some embodiments, the terminator comprises the nucleic acid sequence set forth in SEQ ID NO: 52, with 1, 2, 3, 4, or 5 nucleotide deletions compared to the nucleic acid sequence set forth in SEQ ID NO: 52. In some embodiments, the terminator comprises a nucleic acid sequence at least 85%, 86%, 87%, 88%, 89%, 90%, 91%, 92%, 93%, 94%, 95%, 96%, 97%, 98%, 99%, or 100% identical to the nucleic acid sequence set forth in SEQ ID NO: 52. In some embodiments, the terminator comprises the nucleic acid sequence set forth in SEQ ID NO: 52. In some embodiments, the terminator consists essentially of the nucleic acid sequence set forth in SEQ ID NO: 52. In some embodiments, the terminator consists of the nucleic acid sequence set forth in SEQ ID NO: 52. In some embodiments, the terminator comprises the nucleic acid sequence set forth in SEQ ID NO: 52, modified such that the protein X promoter and coding sequence is removed, and/or all relevant “ATG”s are mutated to “TGG” or “CGG.”

In some embodiments, the terminator is a WPRE comprising the nucleic acid sequence of SEQ ID NO: 53. In some embodiments, the terminator comprises the nucleic acid sequence set forth in SEQ ID NO: 53, with 1, 2, 3, 4, 5, 10, 15, or 20 nucleotide modifications compared to the nucleic acid sequence set forth in SEQ ID NO: 53. In some embodiments, the terminator comprises the nucleic acid sequence set forth in SEQ ID NO: 53, with 1, 2, 3, 4, or 5 nucleotide deletions compared to the nucleic acid sequence set forth in SEQ ID NO: 53. In some embodiments, the terminator comprises a nucleic acid sequence at least 85%, 86%, 87%, 88%, 89%, 90%, 91%, 92%, 93%, 94%, 95%, 96%, 97%, 98%, 99%, or 100% identical to the nucleic acid sequence set forth in SEQ ID NO: 53. In some embodiments, the terminator comprises the nucleic acid sequence set forth in SEQ ID NO: 53. In some embodiments, the terminator consists essentially of the nucleic acid sequence set forth in SEQ ID NO: 53. In some embodiments, the terminator consists of the nucleic acid sequence set forth in SEQ ID NO: 53. In some embodiments, the terminator comprises the nucleic acid sequence set forth in SEQ ID NO: 53, modified such that the protein X promoter and coding sequence is removed, and/or all relevant “ATG”s are mutated to “TGG” or “CGG.”

In some embodiments, the terminator is a WPRE comprising the nucleic acid sequence of SEQ ID NO: 54. In some embodiments, the terminator comprises the nucleic acid sequence set forth in SEQ ID NO: 54, with 1, 2, 3, 4, 5, 10, 15, or 20 nucleotide modifications compared to the nucleic acid sequence set forth in SEQ ID NO: 54. In some embodiments, the terminator comprises the nucleic acid sequence set forth in SEQ ID NO: 54, with 1, 2, 3, 4, or 5 nucleotide deletions compared to the nucleic acid sequence set forth in SEQ ID NO: 54. In some embodiments, the terminator comprises a nucleic acid sequence at least 85%, 86%, 87%, 88%, 89%, 90%, 91%, 92%, 93%, 94%, 95%, 96%, 97%, 98%, 99%, or 100% identical to the nucleic acid sequence set forth in SEQ ID NO: 54. In some embodiments, the terminator comprises the nucleic acid sequence set forth in SEQ ID NO: 54. In some embodiments, the terminator consists essentially of the nucleic acid sequence set forth in SEQ ID NO: 54. In some embodiments, the terminator consists of the nucleic acid sequence set forth in SEQ ID NO: 54. In some embodiments, the terminator comprises the nucleic acid sequence set forth in SEQ ID NO: 54, modified such that the protein X promoter and coding sequence is removed, and/or all relevant “ATG”s are mutated to “TGG” or “CGG.”

In some embodiments, the terminator is a C2 terminator that comprises the nucleic acid sequence of SEQ ID NO: 8. In some embodiments, the terminator comprises the nucleic acid sequence set forth in SEQ ID NO: 8, with 1, 2, 3, 4, or 5 nucleotide modifications compared to the nucleic acid sequence set forth in SEQ ID NO: 8. In some embodiments, the terminator comprises a nucleic acid sequence at least 85%, 86%, 87%, 88%, 89%, 90%, 91%, 92%, 93%, 94%, 95%, 96%, 97%, 98%, 99%, or 100% identical to the nucleic acid sequence set forth in SEQ ID NO: 8. In some embodiments, the terminator comprises the nucleic acid sequence set forth in SEQ ID NO: 8. In some embodiments, the terminator consists essentially of the nucleic acid sequence set forth in SEQ ID NO: 8. In some embodiments, the terminator consists of the nucleic acid sequence set forth in SEQ ID NO: 8.

In some embodiments, the terminator is an alpha 2 globin pause site that comprises the nucleic acid sequence of SEQ ID NO: 49. In some embodiments, the terminator comprises the nucleic acid sequence set forth in SEQ ID NO: 49, with 1, 2, 3, 4, or 5 nucleotide modifications compared to the nucleic acid sequence set forth in SEQ ID NO: 49. In some embodiments, the terminator comprises a nucleic acid sequence at least 85%, 86%, 87%, 88%, 89%, 90%, 91%, 92%, 93%, 94%, 95%, 96%, 97%, 98%, 99%, or 100% identical to the nucleic acid sequence set forth in SEQ ID NO: 49. In some embodiments, the terminator comprises the nucleic acid sequence set forth in SEQ ID NO: 49. In some embodiments, the terminator consists essentially of the nucleic acid sequence set forth in SEQ ID NO: 49. In some embodiments, the terminator consists of the nucleic acid sequence set forth in SEQ ID NO: 49.

In some embodiments, the terminator is a human beta globin CoTC that comprises the nucleic acid sequence of SEQ ID NO: 50. In some embodiments, the terminator comprises the nucleic acid sequence set forth in SEQ ID NO: 50, with 1, 2, 3, 4, or 5 nucleotide modifications compared to the nucleic acid sequence set forth in SEQ ID NO: 50. In some embodiments, the terminator comprises a nucleic acid sequence at least 85%, 86%, 87%, 88%, 89%, 90%, 91%, 92%, 93%, 94%, 95%, 96%, 97%, 98%, 99%, or 100% identical to the nucleic acid sequence set forth in SEQ ID NO: 50. In some embodiments, the terminator comprises the nucleic acid sequence set forth in SEQ ID NO: 50. In some embodiments, the terminator consists essentially of the nucleic acid sequence set forth in SEQ ID NO: 50. In some embodiments, the terminator consists of the nucleic acid sequence set forth in SEQ ID NO: 50.

In some embodiments, the terminator is a mouse beta-major globin pause site that comprises the nucleic acid sequence of SEQ ID NO: 51. In some embodiments, the terminator comprises the nucleic acid sequence set forth in SEQ ID NO: 51, with 1, 2, 3, 4, or 5 nucleotide modifications compared to the nucleic acid sequence set forth in SEQ ID NO: 51. In some embodiments, the terminator comprises the nucleic acid sequence set forth in SEQ ID NO: 51. In some embodiments, the terminator consists essentially of the nucleic acid sequence set forth in SEQ ID NO: 51. In some embodiments, the terminator consists of the nucleic acid sequence set forth in SEQ ID NO: 51.

The nucleic acid sequence of exemplary terminators is provided in Table 5.

TABLE 5
Exemplary Terminators

| | | SEQ |
|---|---|---|
| | | ID |
| Name | Nucleic Acid Sequence | NO |
| C2 | CAGTGCCTCTATCTGGAGGCCAGGTAGGGCTG | 8 |
| | GCCTTGGGGGAGGGGGAGGCCAGAATGACTCC | |
| | AAGAGCTACAGGAAGGCAGGTCAGAGACCCCA | |
| | CTGGACAAACAGTGGCTGGACTCTGCACCATA | |
| | ACACACAATCAACAGGGGAGTGAGCTGG | |
| Safety | AATCAACCTCTGGATTACAAAATTTGTGAAAG | 9 |
| modified | ATTGACTGGTATTCTTAACTATGTTGCTCCTT | |
| WPRE WHV | TTACGCTtgGTGGATACGCTGCTTTAcgGCCT | |
| strain | TTGTATCtgGCTATTGCTTCCCGTATGGCTTT | |
| WHV8 | CATTTTCTCCTCCTTGTATAAATCCTGGTTGC | |
| (Derived | TGTCTCTTTtgGAGGAGTTGTGGCCCGTTGTC | |
| from | AGGCAACGTGGCGTGGTGTGCACTGTGTTTGC | |
| GenBank: | TGACGCAACCCCCACTGGTTGGGGCATTGCCA | |
| J04514.1) | CCACCTGTCAGCTCCTTTCCGGGACTTTCGCT | |
| | TTCCCCCTCCCTATTGCCACGGCGGAACTCAT | |
| | CGCCGCCTGCCTTGCCCGCTGCTGGACAGGGG | |
| | CTCGGCTGTTGGGCACTGACAATTCCGTGGTG | |
| | TTGTC | |
| Non- | AATCAACCTCTGGATTACAAAATTTGTGAAAG | 52 |
| safety | ATTGACTGGTATTCTTAACTATGTTGCTCCTT | |
| modified | TTACGCTATGTGGATACGCTGCTTTAATGCCT | |
| WPRE (WT) | TTGTATCATGCTATTGCTTCCCGTATGGCTTT | |
| WHV | CATTTTCTCCTCCTTGTATAAATCCTGGTTGC | |
| strain | TGTCTCTTTATGAGGAGTTGTGGCCCGTTGTC | |
| WHV8 | AGGCAACGTGGCGTGGTGTGCACTGTGTTTGC | |
| (GenBank: | TGACGCAACCCCCACTGGTTGGGGCATTGCCA | |
| J04514.1) | CCACCTGTCAGCTCCTTTCCGGGACTTTCGCT | |
| Beta | TTCCCCCTCCCTATTGCCACGGCGGAACTCAT | |
| subunit | CGCCGCCTGCCTTGCCCGCTGCTGGACAGGGG | |
| is bold | CTCGGCTGTTGGGCACTGACAATTCCGTGGTG | |
| and | TTGTC | |
| under- | GGGGAAGCTGACGTCC | |
| lined | TTTCCATGGCTGCTCG | |
| | CCTGTGTTGCCACCTG | |
| | GATTCTGCGCGGGACG | |
| | TCCTTCTGCTACGTCC | |
| | CTTCGGCCCTCAATCC | |
| | AGCGGACCTTCCTTCC | |
| | CGCGGCCTGCTGCCGG | |
| | CTCTGCGGCCTCTTCC | |
| | GCGTCTTCGCCTTCGC | |
| | CCTCAGACGAGTCGGA | |
| | TCTCCCTTTGGGCCGC | |
| | CTCCCCGCCTG | |
| Non- | AATCAACCTCTGGATTACAAAATTTGTGAAAG | 53 |
| safety | ATTGACTGATATTCTTAACTATGTTGCTCCTT | |
| modified | TTACGCTGTGTGGATATGCTGCTTTAATGCCT | |
| WPRE (WT) | CTGTATCATGCTATTGCTTCCCGTACGGCTTT | |
| WHV | CGTTTTCTCCTCCTTGTATAAATCCTGGTTGC | |
| strain | TGTCTCTTTATGAGGAGTTGTGGCCCGTTGTC | |
| (GenBank: | CGTCAACGTGGCGTGGTGTGCTCTGTGTTTGC | |
| J02442.1) | TGACGCAACCCCCACTGGCTGGGGCATTGCCA | |
| Beta | CCACCTGTCAACTCCTTTCTGGGACTTTCGCT | |
| subunit | TTCCCCCTCCCGATCGCCACGGCAGAACTCAT | |
| is bold | CGCCGCCTGCCTTGCCCGCTGCTGGACAGGGG | |
| and | CTAGGTTGCTGGGCACTGATAATTCCGTGGTG | |
| under- | TTGTC | |
| lined | GGGGAAGCTGACGTCC | |
| | TTTCCATGGCTGCTCG | |
| | CCTGTGTTGCCAACTG | |
| | GATCCTGCGCGGGACG | |
| | TCCTTCTGCTACGTCC | |
| | CTTCGGCTCTCAATCC | |
| | AGCGGACCTCCCTTCC | |
| | CGAGGCCTTCTGCCGG | |
| | TTCTGCGGCCTCTCCC | |
| | GCGTCTTCGCTTTCGG | |
| | CCTCCGACGAGTCGGA | |
| | TCTCCCTTTGGGCCGC | |
| | CTCCCCGCCTG | |
| Safety | AATCAACCTCTGGATTACAAAATTTGTGAAAG | 54 |
| modified | ATTGACTGATATTCTTAACTATGTTGCTCCTT | |
| WPRE WHV | TTACGCTTGGTGGATATGCTGCTTTACGGCCT | |
| strain | CTGTATCTGGCTATTGCTTCCCGTACGGCTTT | |
| (Derived | CGTTTTCTCCTCCTTGTATAAATCCTGGTTGC | |
| from | TGTCTCTTTTGGAGGAGTTGTGGCCCGTTGTC | |
| GenBank: | CGTCAACGTGGCGTGGTGTGCTCTGTGTTTGC | |
| J02442.1) | TGACGCAACCCCCACTGGCTGGGGCATTGCCA | |
| | CCACCTGTCAACTCCTTTCTGGGACTTTCGCT | |
| | TTCCCCCTCCCGATCGCCACGGCAGAACTCAT | |
| | CGCCGCCTGCCTTGCCCGCTGCTGGACAGGGG | |
| | CTAGGTTGCTGGGCACTGATAATTCCGTGGTG | |
| | TTGTC | |
| alpha 2 | AACATACGCTCTCCATCAAAACAAAACGAAAC | 49 |
| globin | AAAACAAACTAGCAAAATAGGCTGTCCCCAGT | |
| pause | GCAAGTGCAGGTGCCAGAACATTTCTCT | |
| site | | |
| human | CAATAACAAACAAAAAATTAAAAATAGGAAAA | 50 |
| beta | TAAAAAAATTAAAAAGAAGAAAATCCTGCCAT | |
| globin | TTATGCGAGAATTGATGAACCTGGAGGATGTA | |
| CoTC | AAACTAAGAAAAATAAGCCTGACACAAAAAGA | |
| | CAAATACTACACAACCTTGCTCATATGTGAAA | |
| | GATAAAAAAGTCACTCTCATGGAAACAGACAG | |
| | TAGAGGTATGGTTTCCAGGGGTTGGGGGTGGG | |
| | AGAATCAGGAAACTATTACTCAAAGGGTATAA | |
| | AATTTCAGTTATGTGGGATGAATAAATT | |
| mouse | GAAGTAAAGAGTTAGAGTATGGTGAGAAATTA | 51 |
| beta- | TAAACCATCAAAGAAAAAAATACAGGACCCAT | |
| major | AAAGG | |
| globin | | |
| pause | | |
| site | | |

In one aspect, provided herein are polynucleotides that comprise a safety modified WPRE terminator. In some embodiments, the safety modification comprises at least one nucleotide modification. Exemplary modifications include those described by in Schambach A et al., Woodchuck hepatitis virus post-transcriptional regulatory element deleted from X protein and promoter sequences enhances retroviral vector titer and expression, Gene Ther. 2006; 13(7):641-645. doi:10.1038/sj.gt.3302698 (the contents of which are incorporated by reference herein). Exemplary modifications include, but are not limited to, removal of the protein X promoter and coding sequence, and mutation of all relevant “ATG”s to “TGG” or “CGG.”

In some embodiments, the safety modified WPRE comprises the nucleic acid sequence set forth in SEQ ID NO: 8, with 1, 2, 3, 4, or 5 nucleotide modifications compared to the nucleic acid sequence set forth in SEQ ID NO: 8. In some embodiments, the safety modified WPRE comprises the nucleic acid sequence set forth in SEQ ID NO: 8. In some embodiments, the safety modified WPRE consists essentially of the nucleic acid sequence set forth in SEQ ID NO: 8. In some embodiments, the safety modified WPRE consists of the nucleic acid sequence set forth in SEQ ID NO: 8.

### Exemplary PolyA Sequences

Exemplary non-naturally occurring polyA sequences described herein are SynHGH V2 (SEQ ID NO: 7) and SynHGH V3 (SEQ ID NO: 18). In some embodiments, the non-naturally occurring polyA sequence is SynHGH V2. In some embodiments, the non-naturally occurring polyA sequence is SynHGH V3.

In some embodiments, the non-naturally occurring polyA sequence comprises a nucleic acid sequence with at least 85%, 86%, 87%, 88%, 89%, 90%, 91%, 92%, 93%, 94%, 95%, 96%, 97%, 98%, 99%, or 100% identity to the nucleic acid sequence set forth in SEQ ID NO: 7. In some embodiments, the non-naturally occurring polyA sequence comprises the nucleic acid sequence set forth in SEQ ID NO: 7, with 1, 2, 3, 4, 5, 6, 7, 8, 9, or 10 nucleotide modifications. In some embodiments, the non-naturally occurring polyA sequence comprises the nucleic acid sequence set forth in SEQ ID NO: 7. In some embodiments, the non-naturally occurring polyA sequence consists essentially of the nucleic acid sequence set forth in SEQ ID NO: 7. In some embodiments, the non-naturally occurring polyA sequence consists of the nucleic acid sequence set forth in SEQ ID NO: 7.

In some embodiments, the non-naturally occurring polyA sequence comprises a nucleic acid sequence with at least 90%, 91%, 92%, 93%, 94%, 95%, 96%, 97%, 98%, 99%, or 100% identity to the nucleic acid sequence set forth in SEQ ID NO: 18. In some embodiments, the non-naturally occurring polyA sequence comprises the nucleic acid sequence set forth in SEQ ID NO: 18, with 1, 2, 3, 4, 5, 6, 7, 8, 9, or 10 nucleotide modifications. In some embodiments, the non-naturally occurring polyA sequence comprises the nucleic acid sequence set forth in SEQ ID NO: 18. In some embodiments, the non-naturally occurring polyA sequence consists essentially of the nucleic acid sequence set forth in SEQ ID NO: 18. In some embodiments, the non-naturally occurring polyA sequence consists of the nucleic acid sequence set forth in SEQ ID NO: 18.

In some embodiments, the non-naturally occurring polyA sequence comprises a nucleic acid sequence with at least 90%, 91%, 92%, 93%, 94%, 95%, 96%, 97%, 98%, 99%, or 100% identity to the nucleic acid sequence set forth in SEQ ID NO: 10. In some embodiments, the non-naturally occurring polyA sequence comprises the nucleic acid sequence set forth in SEQ ID NO: 10, with 1, 2, 3, 4, 5, 6, 7, 8, 9, or 10 nucleotide modifications. In some embodiments, the non-naturally occurring polyA sequence comprises the nucleic acid sequence set forth in SEQ ID NO: 10. In some embodiments, the non-naturally occurring polyA sequence consists essentially of the nucleic acid sequence set forth in SEQ ID NO: 10. In some embodiments, the non-naturally occurring polyA sequence consists of the nucleic acid sequence set forth in SEQ ID NO: 10.

In some embodiments, the non-naturally occurring polyA sequence comprises a nucleic acid sequence with at least 90%, 91%, 92%, 93%, 94%, 95%, 96%, 97%, 98%, 99%, or 100% identity to the nucleic acid sequence set forth in SEQ ID NO: 11. In some embodiments, the non-naturally occurring polyA sequence comprises the nucleic acid sequence set forth in SEQ ID NO: 11, with 1, 2, 3, 4, 5, 6, 7, 8, 9, or 10 nucleotide modifications. In some embodiments, the non-naturally occurring polyA sequence comprises the nucleic acid sequence set forth in SEQ ID NO: 11. In some embodiments, the non-naturally occurring polyA sequence consists essentially of the nucleic acid sequence set forth in SEQ ID NO: 11. In some embodiments, the non-naturally occurring polyA sequence consists of the nucleic acid sequence set forth in SEQ ID NO: 11.

In some embodiments, the non-naturally occurring polyA sequence comprises a nucleic acid sequence with at least 90%, 91%, 92%, 93%, 94%, 95%, 96%, 97%, 98%, 99%, or 100% identity to the nucleic acid sequence set forth in SEQ ID NO: 12. In some embodiments, the non-naturally occurring polyA sequence comprises the nucleic acid sequence set forth in SEQ ID NO: 12, with 1, 2, 3, 4, 5, 6, 7, 8, 9, or 10 nucleotide modifications. In some embodiments, the non-naturally occurring polyA sequence comprises the nucleic acid sequence set forth in SEQ ID NO: 12. In some embodiments, the non-naturally occurring polyA sequence consists essentially of the nucleic acid sequence set forth in SEQ ID NO: 12. In some embodiments, the non-naturally occurring polyA sequence consists of the nucleic acid sequence set forth in SEQ ID NO: 12.

In some embodiments, the non-naturally occurring polyA sequence comprises a nucleic acid sequence with at least 90%, 91%, 92%, 93%, 94%, 95%, 96%, 97%, 98%, 99%, or 100% identity to the nucleic acid sequence set forth in SEQ ID NO: 19. In some embodiments, the non-naturally occurring polyA sequence comprises the nucleic acid sequence set forth in SEQ ID NO: 19, with 1, 2, 3, 4, 5, 6, 7, 8, 9, or 10 nucleotide modifications. In some embodiments, the non-naturally occurring polyA sequence comprises the nucleic acid sequence set forth in SEQ ID NO: 19. In some embodiments, the non-naturally occurring polyA sequence consists essentially of the nucleic acid sequence set forth in SEQ ID NO: 19. In some embodiments, the non-naturally occurring polyA sequence consists of the nucleic acid sequence set forth in SEQ ID NO: 19.

In some embodiments, the non-naturally occurring polyA sequence comprises a nucleic acid sequence with at least 90%, 91%, 92%, 93%, 94%, 95%, 96%, 97%, 98%, 99%, or 100% identity to the nucleic acid sequence set forth in SEQ ID NO: 20. In some embodiments, the non-naturally occurring polyA sequence comprises the nucleic acid sequence set forth in SEQ ID NO: 20, with 1, 2, 3, 4, 5, 6, 7, 8, 9, or 10 nucleotide modifications. In some embodiments, the non-naturally occurring polyA sequence comprises the nucleic acid sequence set forth in SEQ ID NO: 20. In some embodiments, the non-naturally occurring polyA sequence consists essentially of the nucleic acid sequence set forth in SEQ ID NO: 20. In some embodiments, the non-naturally occurring polyA sequence consists of the nucleic acid sequence set forth in SEQ ID NO: 20.

In some embodiments, the non-naturally occurring polyA sequence comprises a nucleic acid sequence with at least 90%, 91%, 92%, 93%, 94%, 95%, 96%, 97%, 98%, 99%, or 100% identity to the nucleic acid sequence set forth in SEQ ID NO: 21. In some embodiments, the non-naturally occurring polyA sequence comprises the nucleic acid sequence set forth in SEQ ID NO: 21, with 1, 2, 3, 4, 5, 6, 7, 8, 9, or 10 nucleotide modifications. In some embodiments, the non-naturally occurring polyA sequence comprises the nucleic acid sequence set forth in SEQ ID NO: 21. In some embodiments, the non-naturally occurring polyA sequence consists essentially of the nucleic acid sequence set forth in SEQ ID NO: 21. In some embodiments, the non-naturally occurring polyA sequence consists of the nucleic acid sequence set forth in SEQ ID NO: 21.

Exemplary non-naturally occurring polyAs are provided in Table 6.

TABLE 6
SynHGH V2 and SynHGH V2 Non-naturally
occurring PolyAs

| | | SEQ |
|---|---|---|
| | | ID |
| Name | Nucleic Acid Sequence | NO |
| SynHGH | CCTCTCCTGGCCCTGGAAGTTGCCACTCCAGTG | 7 |
| V2 | CCGACCAGCCTTGTCCTAATAAACAAGTTAACA | |
| | ACAATTTTGTCTCGTGTGTTGGAATTTTTTGTG | |
| | TCTCTGGGGTGGAGGGGGGTGGTATGGAGCAAG | |
| | GGG | |
| SynHGH | TTTATTTGTGAAATTTGTGATGCTATTGCTTTA | 18 |
| V3 | TTTGTAACCATTTTATTTGTGAAATTTGTGATG | |
| | CTATTGCTTTATTTGTAACCACAATAAAATTAA | |
| | GTTGCATCATTTTGTCTGACTAGGTGTCCTTCT | |
| | ATAATATTATGGGGTGGAGGGGGGTGGTATGGA | |
| | GCAAGGGG | |
| WPRE | AATCAACCTCTGGATTACAAAATTTGTGAAAGA | 10 |
| SynHGH | TTGACTGGTATTCTTAACTATGTTGCTCCTTTT | |
| V2 | ACGCTTGGTGGATACGCTGCTTTACGGCCTTTG | |
| | TATCTGGCTATTGCTTCCCGTATGGCTTTCATT | |
| | TTCTCCTCCTTGTATAAATCCTGGTTGCTGTCT | |
| | CTTTTGGAGGAGTTGTGGCCCGTTGTCAGGCAA | |
| | CGTGGCGTGGTGTGCACTGTGTTTGCTGACGCA | |
| | ACCCCCACTGGTTGGGGCATTGCCACCACCTGT | |
| | CAGCTCCTTTCCGGGACTTTCGCTTTCCCCCTC | |
| | CCTATTGCCACGGCGGAACTCATCGCCGCCTGC | |
| | CTTGCCCGCTGCTGGACAGGGGCTCGGCTGTTG | |
| | GGCACTGACAATTCCGTGGTGTTGTCCCTCTCC | |
| | TGGCCCTGGAAGTTGCCACTCCAGTGCCGACCA | |
| | GCCTTGTCCTAATAAACAAGTTAACAACAATTT | |
| | TGTCTCGTGTGTTGGAATTTTTTGTGTCTCTGG | |
| | GGTGGAGGGGGGTGGTATGGAGCAAGGGG | |
| SynHGH | CCTCTCCTGGCCCTGGAAGTTGCCACTCCAGTG | 11 |
| V2 | CCGACCAGCCTTGTCCTAATAAACAAGTTAACA | |
| C2 | ACAATTTTGTCTCGTGTGTTGGAATTTTTTGTG | |
| | TCTCTGGGGTGGAGGGGGGTGGTATGGAGCAAG | |
| | GGGCAGTGCCTCTATCTGGAGGCCAGGTAGGGC | |
| | TGGCCTTGGGGGAGGGGGAGGCCAGAATGACTC | |
| | CAAGAGCTACAGGAAGGCAGGTCAGAGACCCCA | |
| | CTGGACAAACAGTGGCTGGACTCTGCACCATAA | |
| | CACACAATCAACAGGGGAGTGAGCTGG | |
| WPRE | AATCAACCTCTGGATTACAAAATTTGTGAAAGA | 12 |
| SynHGH | TTGACTGGTATTCTTAACTATGTTGCTCCTTTT | |
| V2 | ACGCTTGGTGGATACGCTGCTTTACGGCCTTTG | |
| C2 | TATCTGGCTATTGCTTCCCGTATGGCTTTCATT | |
| | TTCTCCTCCTTGTATAAATCCTGGTTGCTGTCT | |
| | CTTTTGGAGGAGTTGTGGCCCGTTGTCAGGCAA | |
| | CGTGGCGTGGTGTGCACTGTGTTTGCTGACGCA | |
| | ACCCCCACTGGTTGGGGCATTGCCACCACCTGT | |
| | CAGCTCCTTTCCGGGACTTTCGCTTTCCCCCTC | |
| | CCTATTGCCACGGCGGAACTCATCGCCGCCTGC | |
| | CTTGCCCGCTGCTGGACAGGGGCTCGGCTGTTG | |
| | GGCACTGACAATTCCGTGGTGTTGTCCCTCTCC | |
| | TGGCCCTGGAAGTTGCCACTCCAGTGCCGACCA | |
| | GCCTTGTCCTAATAAACAAGTTAACAACAATTT | |
| | TGTCTCGTGTGTTGGAATTTTTTGTGTCTCTGG | |
| | GGTGGAGGGGGGTGGTATGGAGCAAGGGG | |
| WPRE | AATCAACCTCTGGATTACAAAATTTGTGAAAGA | 19 |
| SynHGH | TTGACTGGTATTCTTAACTATGTTGCTCCTTTT | |
| V3 | ACGCTTGGTGGATACGCTGCTTTACGGCCTTTG | |
| | TATCTGGCTATTGCTTCCCGTATGGCTTTCATT | |
| | TTCTCCTCCTTGTATAAATCCTGGTTGCTGTCT | |
| | CTTTTGGAGGAGTTGTGGCCCGTTGTCAGGCAA | |
| | CGTGGCGTGGTGTGCACTGTGTTTGCTGACGCA | |
| | ACCCCCACTGGTTGGGGCATTGCCACCACCTGT | |
| | CAGCTCCTTTCCGGGACTTTCGCTTTCCCCCTC | |
| | CCTATTGCCACGGCGGAACTCATCGCCGCCTGC | |
| | CTTGCCCGCTGCTGGACAGGGGCTCGGCTGTTG | |
| | GGCACTGACAATTCCGTGGTGTTGTCTTTATTT | |
| | GTGAAATTTGTGATGCTATTGCTTTATTTGTAA | |
| | CCATTTTATTTGTGAAATTTGTGATGCTATTGC | |
| | TTTATTTGTAACCACAATAAAATTAAGTTGCAT | |
| | CATTTTGTCTGACTAGGTGTCCTTCTATAATAT | |
| | TATGGGGTGGAGGGGGGTGGTATGGAGCAAGGG | |
| | G | |
| SynHGH | TTTATTTGTGAAATTTGTGATGCTATTGCTTTA | 20 |
| V3 | TTTGTAACCATTTTATTTGTGAAATTTGTGATG | |
| C2 | CTATTGCTTTATTTGTAACCACAATAAAATTAA | |
| | GTTGCATCATTTTGTCTGACTAGGTGTCCTTCT | |
| | ATAATATTATGGGGTGGAGGGGGGTGGTATGGA | |
| | GCAAGGGGCAGTGCCTCTATCTGGAGGCCAGGT | |
| | AGGGCTGGCCTTGGGGGAGGGGGAGGCCAGAAT | |
| | GACTCCAAGAGCTACAGGAAGGCAGGTCAGAGA | |
| | CCCCACTGGACAAACAGTGGCTGGACTCTGCAC | |
| | CATAACACACAATCAACAGGGGAGTGAGCTGG | |
| WPRE | AATCAACCTCTGGATTACAAAATTTGTGAAAGA | 21 |
| SynHGH | TTGACTGGTATTCTTAACTATGTTGCTCCTTTT | |
| V3 | ACGCTTGGTGGATACGCTGCTTTACGGCCTTTG | |
| C2 | TATCTGGCTATTGCTTCCCGTATGGCTTTCATT | |
| | TTCTCCTCCTTGTATAAATCCTGGTTGCTGTCT | |
| | CTTTTGGAGGAGTTGTGGCCCGTTGTCAGGCAA | |
| | CGTGGCGTGGTGTGCACTGTGTTTGCTGACGCA | |
| | ACCCCCACTGGTTGGGGCATTGCCACCACCTGT | |
| | CAGCTCCTTTCCGGGACTTTCGCTTTCCCCCTC | |
| | CCTATTGCCACGGCGGAACTCATCGCCGCCTGC | |
| | CTTGCCCGCTGCTGGACAGGGGCTCGGCTGTTG | |
| | GGCACTGACAATTCCGTGGTGTTGTCTTTATTT | |
| | GTGAAATTTGTGATGCTATTGCTTTATTTGTAA | |
| | CCATTTTATTTGTGAAATTTGTGATGCTATTGC | |
| | TTTATTTGTAACCACAATAAAATTAAGTTGCAT | |
| | CATTTTGTCTGACTAGGTGTCCTTCTATAATAT | |
| | TATGGGGTGGAGGGGGGTGGTATGGAGCAAGGG | |
| | GCAGTGCCTCTATCTGGAGGCCAGGTAGGGCTG | |
| | GCCTTGGGGGAGGGGGAGGCCAGAATGACTCCA | |
| | AGAGCTACAGGAAGGCAGGTCAGAGACCCCACT | |
| | GGACAAACAGTGGCTGGACTCTGCACCATAACA | |
| | CACAATCCAACAGGGGAGTGAGTGG | |

Scanning and Removal of miRNA Binding Sites

In some embodiments, the non-naturally occurring polyA sequences described herein are scanned for predicted miRNA binding sites (e.g., human miRNA binding sites). In some embodiments, each predicted miRNA binding site in a non-naturally occurring polyA sequence described herein are removed, e.g., through modification of one or more nucleotides of the miRNA binding site. mRNA binding sites can be predicted from a nucleic acid sequence through software programs known to those of ordinary skill in the art, e.g., miRBD miRNA target predictor tool (http://mirdb.org/custom.html).

### Vectors

In one aspect, provided herein are vectors that comprise a non-naturally occurring polyA sequence described herein. Any suitable vector can be utilized, including, e.g., recombinant viral vectors and non-viral vectors (e.g., plasmid). In some embodiments, the vector is a non-viral vector. In some embodiments, the non-viral vector is a plasmid. In some embodiments, the vector is a recombinant viral vector. In some embodiments, the recombinant viral vector is an adeno-associated virus (AAV) vector.

In certain embodiments, the vector is a recombinant AAV (rAAV) vector. In certain embodiments, the rAAV vector comprises from 5′ to 3′: a transcriptional regulatory element (TRE), a transgene, and a non-naturally occurring polyA (e.g., as described herein). In certain embodiments, the rAAV vector comprises from 5′ to 3′: a TRE, an intron, a transgene, and a non-naturally occurring polyA sequence (e.g., as described herein). In certain embodiments, the rAAV vectors disclosed herein further comprise a 5′ inverted terminal repeat (5′ ITR) nucleotide sequence 5′ of the TRE, and a 3′ inverted terminal repeat (3′ ITR) nucleotide sequence 3′ of the polyadenylation sequence associated with a transgene. ITR sequences from any AAV serotype or variant thereof can be used in the rAAV genomes disclosed herein. The 5′ and 3′ ITR can be from an AAV of the same serotype or from AAVs of different serotypes.

In some embodiments, the vector is suitable for use in genomic editing of a cell (editing vectors). In some embodiments, the vector is suitable for use in gene therapy (non-editing vectors).

In some embodiments, the vector comprises a transgene. In some embodiments, the transgene encodes a target protein or functional fragment or variant thereof. In some embodiments, the transgene encodes phenylalanine hydroxylase (PAH), arylsulfatase A (ARSA), Frataxin (FXN), glucose-6-phosphatase, or human factor IX (FIX).

In some embodiments, the transgene encodes a polypeptide that is useful to treat a disease or disorder in a subject. Suitable polypeptides include, without limitation, β-globin, hemoglobin, tissue plasminogen activator, and coagulation factors, such as Factor VIII, Factor IX, Factor X; colony stimulating factors (CSF); interleukins, such as IL-1, IL-2, IL-3, IL-4, IL-5, IL-6, IL-7, IL-8, IL-9; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-a receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as a-glucosidase, imiglucerase, β-glucocerebrosidase, and alglucerase; enzyme activators, such as tissue plasminogen activator; chemokines, such as IP-10, monokine induced by interferon-gamma (Mig), Groα/IL-8, RANTES, MIP-1a, MIP-1β, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotropin; fibrin; hirudin; IL-1 receptor antagonists; ciliary neurotrophic factor (CNTF); brain-derived neurotrophic factor (BDNF); neurotrophins 3 and 4/5 (NT-3 and -4/5); glial cell derived neurotrophic factor (GDNF); aromatic amino acid decarboxylase (AADC); dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes, such as iduronate-2-sulfatase (I2S), and arylsulfatase A; and mitochondrial proteins, such as frataxin.

In certain embodiments, the transgene encodes a protein that may be defective in one or more lysosomal storage diseases. Suitable proteins include, without limitation, α-sialidase, cathepsin A, α-mannosida se, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formyl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, capthepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN3, CLN 6, CLN 8, LYST, MYOV, RAB27A, melanophilin, and AP3 β-subunit.

In certain embodiments, the transgene encodes an antibody or a fragment thereof (e.g., a Fab, scFv, or full-length antibody). Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, teprotumumab, eculizumab, and ravulizumab.

In certain embodiments, the transgene encodes a nuclease. Suitable nucleases include, without limitation, zinc fingers nucleases (ZFN) (see e.g., Porteus, and Baltimore (2003) Science 300: 763; Miller et al. (2007) Nat. Biotechnol. 25:778-785; Sander et al. (2011) Nature Methods 8:67-69; and Wood et al. (2011) Science 333:307, each of which is hereby incorporated by reference in its entirety), transcription activator-like effectors nucleases (TALEN) (see e.g., Wood et al. (2011) Science 333:307; Boch et al. (2009) Science 326:1509-1512; Moscou and Bogdanove (2009) Science 326; 1501; Christian et al. (2010) Genetics 186:757-761; Miller et al. (2011) Nat. Biotechnol. 29:143-148; Zhang et al. (2011) Nat. Biotechnol. 29:149-153; and Reyon et al. (2012) Nat. Biotechnol. 30(5): 460-465, each of which is hereby incorporated by reference in its entirety), homing endonucleases, meganucleases (see, e.g., U.S. Patent Publication No. US 2014/0121115, which is hereby incorporated by reference in its entirety), and RNA-guided nucleases (see e.g., Makarova et al. (2018) The CRISPR Journal 1(5): 325-336; and Adli (2018) Nat. Communications 9:1911, each of which is hereby incorporated by reference in its entirety).

In certain embodiments, the transgene encodes an RNA-guided nuclease. Suitable RNA-guided nucleases include, without limitation, Class I and Class II clustered regularly interspaced short palindromic repeats (CRISPR)-associated nucleases. Class I is divided into types I, III, and IV, and includes, without limitation, type I (Cas3), type I-A (Cas8a, Cas5), type I-B (Cas8b), type I-C(Cas8c), type 1-D (Cas10d), type I-E (Cse1, Cse2), type I-F (Csy1, Csy2, Csy3), type I-U (GSU0054), type III (Cas10), type III-A (Csm2), type III-B (Cmr5), type III-C (Csx10 or Csx11), type III-D (Csx10), and type IV (Csf1). Class II is divided into types II, V, and VI, and includes, without limitation, type II (Cas9), type II-A (Csn2), type II-B (Cas4), type V (Cpf1, C2c1, C2c3), and type VI (Cas13a, Cas13b, Cas13c). RNA-guided nucleases also include naturally-occurring Class II CRISPR nucleases such as Cas9 (Type II) or Cas12a/Cpf1 (Type V), as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the transgene encodes reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In some embodiments, the vector further comprises a TRE. In some embodiments, the TRE comprises a promoter sequence. In some embodiments, the TRE comprises a promoter and an enhancer sequence. Any suitable promoter can be utilized, and determined by a person of ordinary skill in the art from known promoters.

In some embodiments, the TRE is active in any mammalian cell (e.g., human cell). In some embodiments, the TRE is active in a broad range of mammalian (e.g., human) cells. In some embodiments, the TRE is a tissue-specific TRE, i.e., it is active in specific tissue(s) and/or organ(s). A tissue-specific TRE comprises one or more tissue-specific promoter and/or enhancer elements. A skilled artisan would appreciate that tissue-specific promoter and/or enhancer elements can be isolated from genes specifically expressed in the tissue by methods well known in the art.

### Exemplary Methods of Use

In one aspect, provided herein are methods of modifying a cell, comprising introducing the polynucleotide comprising a non-naturally occurring polyA sequence described herein (e.g., a vector described herein), into the cell. In some embodiments, the method comprises the in vivo modification of a cell. In some embodiments, the method comprises the in vitro modification of a cell. In some embodiments, the method comprises the ex vivo modification of a cell.

Any suitable cell can be modified, and readily identified by a person of ordinary skill in the art. For example, the cells can be human or non-human animal. A broad range of cells can be targeted for modification or a narrow subset of cells (e.g., a liver or blood cell).

The polynucleotide can be introduced into the cell in any number of suitable manners known to a person of skill in the art. For example, a polynucleotide containing the non-naturally occurring polyA can be transfected into a cell by any suitable transfection method (e.g., electroporation). The polynucleotide containing the non-naturally occurring polyA can be incorporated into a vector (e.g., a vector described herein) and transfected or transduced into a cell.

In some embodiments, the modified cells express a transgene encoded by a vector introduced into the cell. In some embodiments, the modified cells are genetically modified. In some embodiments, the modified cells are genetically modified such that a transgene is inserted into the genome.

In one aspect, provided herein are methods of treating or preventing a disease or disorder by administering a polynucleotide described herein or vector described herein to a human subject in need thereof. In some embodiments, the administration mediates modification of a population of cells in the human body. In some embodiments, the modification is a genetic modification. In some embodiments, the modified cells express a transgene that is not inserted into the genome. In some embodiments, the modification is not a genetic modification. In some embodiments, the modified cells express a transgene that is inserted into the genome. Any disease or disorder can be treated or prevented that would benefit from expression of the transgene. Exemplary transgenes include, but are not limited to, phenylalanine hydroxylase (PAH), arylsulfatase A (ARSA), Frataxin (FXN), glucose-6-phosphatase, and human factor IX (FIX).

### Examples

### Example 1. Construction of Non-Naturally Occurring PolyA

The non-naturally occurring polyA sequences, SynHGH V2 and SynHGH V3, were constructed as described below. The polyA sequences were cloned into the PGK promoter-driven, luciferase-expressing plasmid pGL4.53, obtained from Promega (catalog #: E5011). In pGL4.53, an SV40 late polyA signal is used to terminate luciferase transcription. For the cloning, the SV40 late polyA signal was swapped out with the SynHVH-V2 and SynHGH-V3 sequences via Gibson assembly. Briefly, a linear PCR product of the full vector minus the polyA sequence was created. Primers used for linearizing the pGL4.53 plasmid are described in Table 7. Double-stranded DNA fragments containing the SynHGH-V2 and SynHGH-V3 sequences, with additional 5′ and 3′ sequences homologous to the ends of the linearized pGL4.53 (Gibson tags) were obtained. The 5′ and 3′ overlap sequences used for Gibson assembly are described in Table 8. Gibson assembly was then carried out, competent cells were transformed with the assembled vector, plated on ampicillin containing plates, and grown overnight. Individual colonies were picked, miniprepped, and screened for the correct insert (SynHGH-V2 or SynHGH-V3 polyA) sequence and intact luciferase coding sequence by Sanger sequencing. Sequence-confirmed plasmids were used for in vitro expression analysis in Example 2.

SynHGH-V2 and SynHGH-V3 were cloned into other luciferase-expressing plasmids in order to compare expression with different promoters. The plasmids were cloned using the same method described above, wherein the plasmid was linearized by PCR and the insert (polyA) was inserted by Gibson assembly. New Gibson tags were generated by performing PCR with primers containing 5′ overhangs of the desired Gibson tag sequence. The primers amplified the insert while also adding on the overhang sequences to the ends of the amplicon, producing inserts that could be assembled into the desired vectors.

TABLE 7
Primer Sequences

| | | SEQ |
|---|---|---|
| | | ID |
| Primer | Nucleic Acid Sequence | NO |
| luc2-Gibson-F | AAATCGATAAGGATCCGTCGACCGATGCCC | 43 |
| luc2-Gibson-R | TTACACGGCGATCTTGCCGCCCTTCTTGGC | 44 |

TABLE 8
5′ and 3′ overlap sequences

| | | SEQ |
|---|---|---|
| | | ID |
| Gibson Tag | Nucleic Acid Sequence | NO |
| 5′ Gibson tag | GAAGGGCGGCAAGATCGCCGTGTAA | 45 |
| 3′ Gibson tag | AAATCGATAAGGATCCGTCGACCGATGCC | 46 |

### Non-Naturally Occurring PolyA SynHGH V2

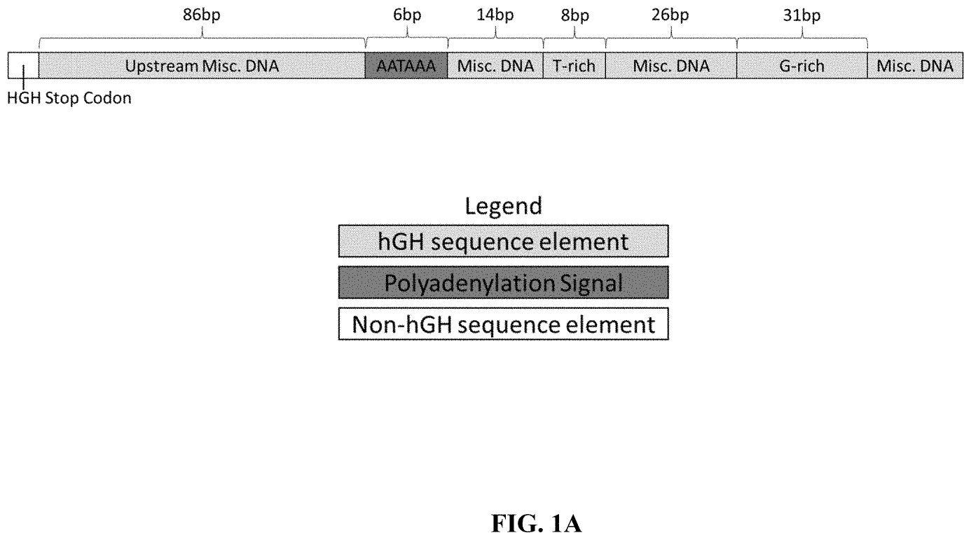

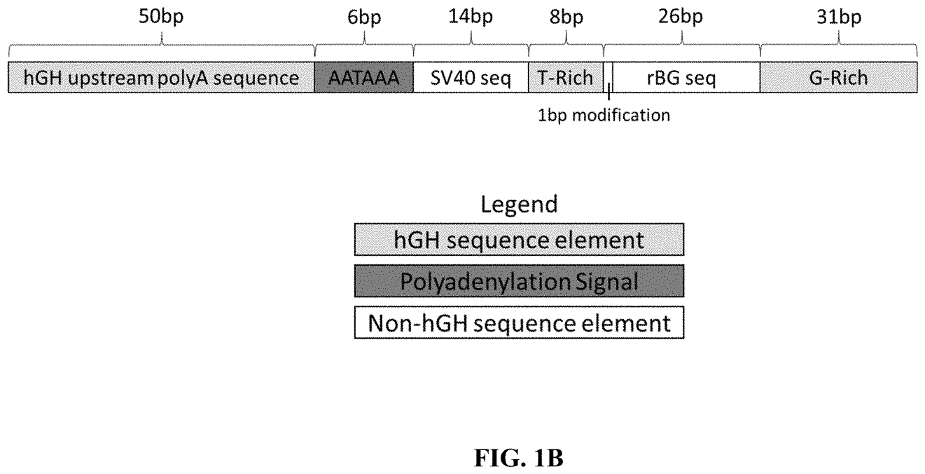

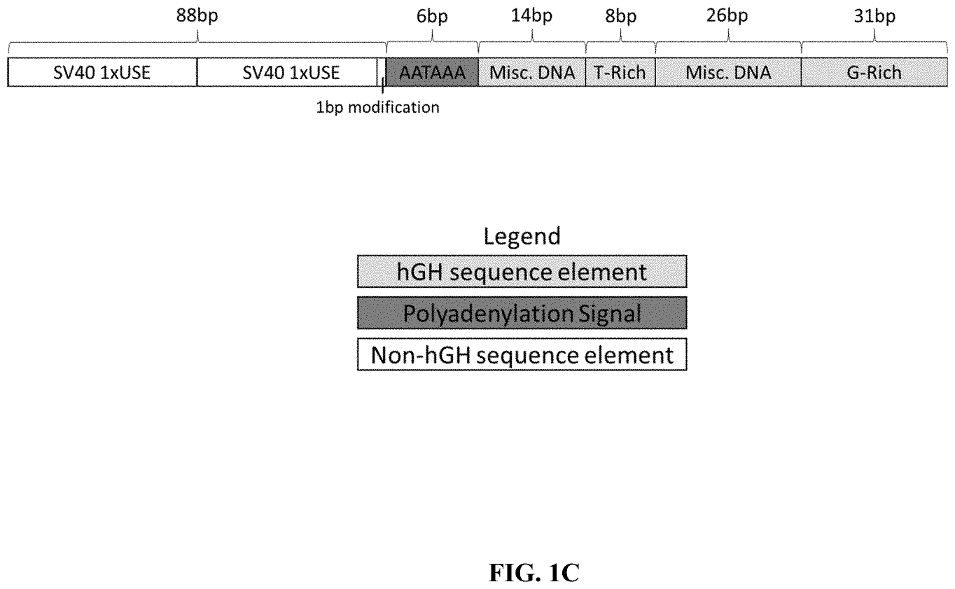

As shown in FIG. 1B, from 5′ to 3′ the SynHGH V2 non-naturally occurring polyA sequence comprises the 50 bp sequence of the hGH gene polyA found upstream of the consensus polyA signal sequence, the consensus polyA signal of hGH, an SV40 late gene polyA sequence that comprises the first 14 bp following the polyA signal sequence of the naturally occurring SV40 late gene polyA sequence, a GT rich region derived from the hGH polyA sequence, a 25 bp intervening sequence derived from the RBG gene polyA sequence that corresponds to bp 24-48 downstream of the polyA signal of the naturally occurring RBG gene polyA sequence, and second GT rich region derived from the naturally occurring hGH polyA sequence. FIG. 1A shows the naturally occurring polyA sequence of the hGH gene. The non-naturally occurring polyA sequence was designed to maintain the respective spacing of the polyA signal sequence and the GT rich regions (a first 6 bp GT rich region, and two closely spaced G-rich regions which together are 31 bp) of the naturally occurring hGH polyA sequence. The sequence of the naturally occurring hGH gene polyA downstream of the last GT rich region was excluded from the SynHGH V2 non-naturally occurring polyA sequence. The RBG downstream sequence element incorporated into the SynHGH V2 non-naturally occurring polyA is known to be important to the function of RBG polyA. See e.g., Levitt et al., Definition of an efficient synthetic poly(A) site, Genes & Dev. 1989. 3: 1019-1025. The sequence of the SynHGH V2 non-naturally occurring polyA was analyzed for miRNA targets using the miRBD miRNA target predictor tool (http://mirdb.org/custom.html). One nucleotide was changed (79A>C) in order to remove two miRNA binding sites. The nucleic acid sequence of the non-naturally occurring polyA SynHGH V2 is provided in Table 9 along with the indicated component nucleic acid sequences.

TABLE 9
SynHGH V2 Non-naturally occurring PolyA

| | | SEQ |
|---|---|---|
| | | NO |
| Name | Nucleic Acid Sequence | ID |
| SynHGH | CCTCTCCTGGCCCTGGAAGTTGCCACTCCA | 7 |
| V2 | GTGCCGACCAGCCTTGTCCTAATAAACAAG | |
| | TTAACAACAATTTTGTCTCGTGTGTTGGAA | |
| | TTTTTTGTGTCTCTGGGGTGGAGGGGGGTG | |
| | GTATGGAGCAAGGGG | |
| PolyA | AATAAA | 1 |
| signal | | |
| sequence | | |
| SynHGH | TTTTGTCT | 2 |
| V2 | | |
| T rich | | |
| region | | |
| SynHGH | GGGGTGGAGGGGGGTGGTATGGAGCAAGGGG | 3 |
| V2 | | |
| G rich | | |
| region | | |
| SynHGH | CAAGTTAACAACAA | 4 |
| V2 | | |
| SV40 | | |
| sequence | | |
| SynHGH | CGTGTGTTGGAATTTTTTGTGTCTCT | 5 |
| V2 | | |
| RBG | | |
| region | | |
| SynHGH | CCTCTCCTGGCCCTGGAAGTTGCCACTCCAG | 6 |
| V2 | TGCCGACCAGCCTTGTCCT | |
| hGH | | |
| upstream | | |
| sequence | | |
| element | | |

### Non-Naturally Occurring PolyA SynHGH V3

As shown in FIG. 1C, from 5′ to 3′ the non-naturally occurring polyA sequence termed SynHGH V3 comprises two copies of the upstream sequence element (USE) derived from the SV40 late gene polyA which comprises the 44 bp sequence which is found upstream of the naturally occurring SV40 late gene polyA signal sequence, the consensus polyA signal sequence of hGH, and the sequence of the hGH polyA sequence that corresponds to the sequence downstream of the polyA signal sequence of the hGH polyA sequence (this region contains to GT rich regions separated by an intervening sequence). FIG. 1A shows the naturally occurring polyA sequence of the hGH gene. The sequence of the SynHGH V3 non-naturally occurring polyA was analyzed for miRNA targets using the miRBD miRNA target predictor tool (http://mirdb.org/custom.html). The nucleic acid sequence of the non-naturally occurring polyA SynHGH V3 is provided in Table 10 along with the indicated component nucleic acid sequences.

TABLE 10
SynHGH V3 Non-naturallv occurring PolyA

| | | SEQ |
|---|---|---|
| | | ID |
| Name | Nucleic Acid Sequence | NO |
| SynHGH | TTTATTTGTGAAATTTGTGATGCTATTGCTTT | 18 |
| V3 | ATTTGTAACCATTTTATTTGTGAAATTTGTGA | |
| | TGCTATTGCTTTATTTGTAACCACAATAAAAT | |
| | TAAGTTGCATCATTTTGTCTGACTAGGTGTCC | |
| | TTCTATAATATTATGGGGTGGAGGGGGGTGGT | |
| | ATGGAGCAAGGGG | |
| SV40 | TTTATTTGTGAAATTTGTGATGCTATTGCTTT | 13 |
| 1X | ATTTGTAACCAC | |
| (with | | |
| 3′ T | | |
| to C | | |
| modifi- | | |
| cation) | | |
| SV40 | TTTATTTGTGAAATTTGTGATGCTATTGCTTT | 14 |
| 1X | ATTTGTAACCAT | |
| (with- | | |
| out | | |
| 3′ T | | |
| to C | | |
| modifi- | | |
| cation) | | |
| SV40 | TTTATTTGTGAAATTTGTGATGCTATTGCTTT | 15 |
| 2X | ATTTGTAACCATTTTATTTGTGAAATTTGTGA | |
| (with | TGCTATTGCTTTATTTGTAACCAC | |
| 3′ T | | |
| to C | | |
| modifi- | | |
| cation) | | |
| SV40 | TTTATTTGTGAAATTTGTGATGCTATTGCTTT | 16 |
| 2X | ATTTGTAACCATTTTATTTGTGAAATTTGTGA | |
| (with- | TGCTATTGCTTTATTTGTAACCAT | |
| out | | |
| 3′ T | | |
| to C | | |
| modifi- | | |
| cation) | | |
| SynHGH | ATTAAGTTGCATCATTTTGTCTGACTAGGTGT | 17 |
| V3 | CCTTCTATAATATTATGGGGTGGAGGGGGGTG | |
| hGH | GTATGGAGCAAGGGG | |
| PolyA | | |

### Example 2. Evaluation of Gene Expression Using Vectors with Non-Naturally Occurring PolyA

The SynHGH V2 and SynHGH V3 non-naturally occurring polyA sequences described in Example 1 were incorporated into a gene expression vector encoding a luciferase reporter protein and a promoter (G6PC, LP1, or PGK). The vectors were introduced into cultured cells (Huh7 or HepG2) and expression of the luciferase reporter protein analyzed. Briefly, cells at ˜70-90% confluency were co-transfected in a 96-well plate with two plasmids using Lipofectamine 2000:1) 99 ng of Firefly luciferase-expressing plasmid (with variable polyA/terminator), and 2) 1 ng Nanoluciferase-expressing plasmid (constant well-to-well normalization control for transfection efficiency). After approximately 72 hours, cells were assayed using the Nano-Glo Dual Luciferase Reporter Assay kit from Promega (catalog #: 1610) per the standard instructions. For each well, luminescence levels of firefly luciferase and nanoluciferase were measured individually using a plate reader. The reporter gene was firefly luciferase for all constructs tested (with a different polyA/terminator depending on the experimental group). The sequences of the pGL4.53 firefly luciferase and codon optimized firefly luciferase sequence are provided in Table 11.

TABLE 11
pGL4.53 firefly luciferase and codon optimized
firefly luciferase sequence

| | | SEQ |
|---|---|---|
| Firefly | | ID |
| Luciferase | Nucleic Acid Sequence | NO |
| pGL4.53 | ATGGAAGATGCCAAAAACATTAAGAAGGG | 47 |
| firefly | CCCAGCGCCATTCTACCCACTCGAAGACG | |
| luciferase | GGACCGCCGGCGAGCAGCTGCACAAAGCC | |
| | ATGAAGCGCTACGCCCTGGTGCCCGGCAC | |
| | CATCGCCTTTACCGACGCACATATCGAGG | |
| | TGGACATTACCTACGCCGAGTACTTCGAG | |
| | ATGAGCGTTCGGCTGGCAGAAGCTATGAA | |
| | GCGCTATGGGCTGAATACAAACCATCGGA | |
| | TCGTGGTGTGCAGCGAGAATAGCTTGCAG | |
| | TTCTTCATGCCCGTGTTGGGTGCCCTGTT | |
| | CATCGGTGTGGCTGTGGCCCCAGCTAACG | |
| | ACATCTACAACGAGCGCGAGCTGCTGAAC | |
| | AGCATGGGCATCAGCCAGCCCACCGTCGT | |
| | ATTCGTGAGCAAGAAAGGGCTGCAAAAGA | |
| | TCCTCAACGTGCAAAAGAAGCTACCGATC | |
| | ATACAAAAGATCATCATCATGGATAGCAA | |
| | GACCGACTACCAGGGCTTCCAAAGCATGT | |
| | ACACCTTCGTGACTTCCCATTTGCCACCC | |
| | GGCTTCAACGAGTACGACTTCGTGCCCGA | |
| | GAGCTTCGACCGGGACAAAACCATCGCCC | |
| | TGATCATGAACAGTAGTGGCAGTACCGGA | |
| | TTGCCCAAGGGCGTAGCCCTACCGCACCG | |
| | CACCGCTTGTGTCCGATTCAGTCATGCCC | |
| | GCGACCCCATCTTCGGCAACCAGATCATC | |
| | CCCGACACCGCTATCCTCAGCGTGGTGCC | |
| | ATTTCACCACGGCTTCGGCATGTTCACCA | |
| | CGCTGGGCTACTTGATCTGCGGCTTTCGG | |
| | GTCGTGCTCATGTACCGCTTCGAGGAGGA | |
| | GCTATTCTTGCGCAGCTTGCAAGACTATA | |
| | AGATTCAATCTGCCCTGCTGGTGCCCACA | |
| | CTATTTAGCTTCTTCGCTAAGAGCACTCT | |
| | CATCGACAAGTACGACCTAAGCAACTTGC | |
| | ACGAGATCGCCAGCGGCGGGGCGCCGCTC | |
| | AGCAAGGAGGTAGGTGAGGCCGTGGCCAA | |
| | ACGCTTCCACCTACCAGGCATCCGCCAGG | |
| | GCTACGGCCTGACAGAAACAACCAGCGCC | |
| | ATTCTGATCACCCCCGAAGGGGACGACAA | |
| | GCCTGGCGCAGTAGGCAAGGTGGTGCCCT | |
| | TCTTCGAGGCTAAGGTGGTGGACTTGGAC | |
| | ACCGGTAAGACACTGGGTGTGAACCAGCG | |
| | CGGCGAGCTGTGCGTCCGTGGCCCCATGA | |
| | TCATGAGCGGCTACGTTAACAACCCCGAG | |
| | GCTACAAACGCTCTCATCGACAAGGACGG | |
| | CTGGCTGCACAGCGGCGACATCGCCTACT | |
| | GGGACGAGGACGAGCACTTCTTCATCGTG | |
| | GACCGGCTGAAGAGCCTGATCAAATACAA | |
| | GGGCTACCAGGTAGCCCCAGCCGAACTGG | |
| | AGAGCATCCTGCTGCAACACCCCAACATC | |
| | TTCGACGCCGGGGTCGCCGGCCTGCCCGA | |
| | CGACGATGCCGGCGAGCTGCCCGCCGCAG | |
| | TCGTCGTGCTGGAACACGGTAAAACCATG | |
| | ACCGAGAAGGAGATCGTGGACTATGTGGC | |
| | CAGCCAGGTTACAACCGCCAAGAAGCTGC | |
| | GCGGTGGTGTTGTGTTCGTGGACGAGGTG | |
| | CCTAAAGGACTGACCGGCAAGTTGGACGC | |
| | CCGCAAGATCCGCGAGATTCTCATTAAGG | |
| | CCAAGAAGGGCGGCAAGATCGCCGTGTAA | |
| Codon | ATGGAGGATGCCAAGAATATTAAGAAAGG | 48 |
| optimized | CCCTGCCCCATTCTACCCTCTGGAAGATG | |
| firefly | GCACTGCTGGAGAGCAACTGCACAAGGCC | |
| luciferase | ATGAAGTCCTATGCCCTGGTCCCTGGCAC | |
| | CATTGCCTTCACTGATGCTCACATTGAGG | |
| | TGGACATCACCTATGCTGAATACTTTGAG | |
| | ATGTCTGTGAGGCTGGCAGAAGCCATGAA | |
| | AAGATATGGACTGAACACCAACCACAGGA | |
| | TTGTGGTGTGCTCTGAGAACTCTCTCCAG | |
| | TTCTTCATGCCTGTGTTAGGAGCCCTGTT | |
| | CATTGGAGTGGCTGTGGCCCCTGCCAATG | |
| | ACATCTACAATGAGAGAGAGCTCCTGAAC | |
| | AGCATGGGCATCAGCCAGCCAACTGTGGT | |
| | CTTTGTGAGCAAGAAGGGCCTGCAAAAGA | |
| | TCCTGAATGTGCAGAAGAAGCTGCCCATC | |
| | ATCCAGAAGATCATCATCATGGACAGCAA | |
| | GACTGACTACCAGGGCTTCCAGAGCATGT | |
| | ATACCTTTGTGACCAGCCACTTACCCCCT | |
| | GGCTTCAATGAGTATGACTTTGTGCCTGA | |
| | GAGCTTTGACAGGGACAAGACCATTGCTC | |
| | TGATTATGAACAGCTCTGGCTCCACTGGA | |
| | CTGCCCAAAGGTGTGGCTCTGCCCCACAG | |
| | AACTGCTTGTGTGAGATTCAGCCATGCCA | |
| | GAGACCCCATCTTTGGCAACCAGATCATC | |
| | CCTGACACTGCCATCCTGTCTGTGGTTCC | |
| | ATTCCATCATGGCTTTGGCATGTTCACAA | |
| | CACTGGGGTACCTGATCTGTGGCTTCAGA | |
| | GTGGTGCTGATGTATAGGTTTGAGGAGGA | |
| | GCTGTTTCTGAGGAGCCTACAAGACTACA | |
| | AGATCCAGTCTGCCCTGCTGGTGCCCACT | |
| | CTGTTCAGCTTCTTTGCCAAGAGCACCCT | |
| | CATTGACAAGTATGACCTGAGCAACCTGC | |
| | ATGAGATTGCCTCTGGAGGAGCACCCCTG | |
| | AGCAAGGAGGTGGGTGAGGCTGTGGCAAA | |
| | GAGGTTCCATCTCCCAGGAATCAGACAGG | |
| | GCTATGGCCTGACTGAGACCACCTCTGCC | |
| | ATCCTCATCACCCCTGAAGGAGATGACAA | |
| | GCCTGGTGCTGTGGGCAAGGTGGTTCCCT | |
| | TTTTTGAGGCCAAGGTGGTGGACCTGGAC | |
| | ACTGGCAAGACCCTGGGAGTGAACCAGAG | |
| | GGGTGAGCTGTGTGTGAGGGGTCCCATGA | |
| | TCATGTCTGGCTATGTGAACAACCCTGAG | |
| | GCCACCAATGCCCTGATTGACAAGGATGG | |
| | CTGGCTGCACTCTGGTGACATTGCCTACT | |
| | GGGATGAGGATGAGCACTTTTTCATTGTG | |
| | GACAGGCTGAAGAGCCTCATCAAGTACAA | |
| | AGGCTACCAAGTGGCACCTGCTGAGCTAG | |
| | AGAGCATCCTGCTCCAGCACCCCAACATC | |
| | TTTGATGCTGGTGTGGCTGGCCTGCCTGA | |
| | TGATGATGCTGGAGAGCTGCCTGCTGCTG | |
| | TTGTGGTTCTGGAGCATGGAAAGAGCATG | |
| | ACTGAGAAGGAGATTGTGGACTATGTGGC | |
| | CAGTCAGGTGACCACTGCCAAGAAGCTGA | |
| | GGGGAGGTGTGGTGTTTGTGGATGAGGTG | |
| | CCAAAGGGTCTGACTGGCAAGCTGGATGC | |
| | CAGAAAGATCAGAGAGATCCTGATCAAGG | |
| | CCAAGAAGGGTGGCAAAATCGCCGTCTAG | |

For each well, the ratio of firefly luciferase to nanoluciferase was calculated, providing a relative expression level for each transfected well (normalized for transfection efficiency). Values for the plate were normalized to the expression values of a single experimental group (typically the SV40 group) in order to allow comparison between different plates (cell types).

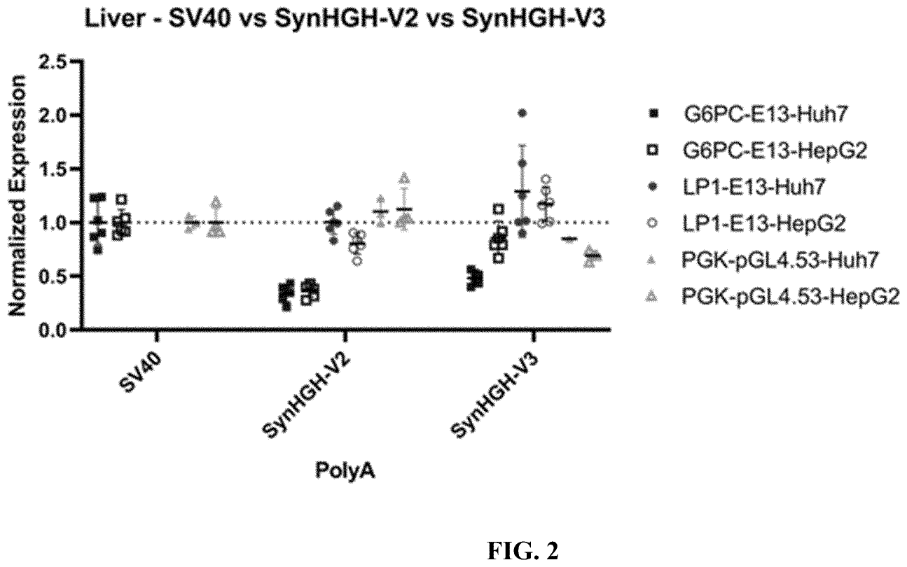

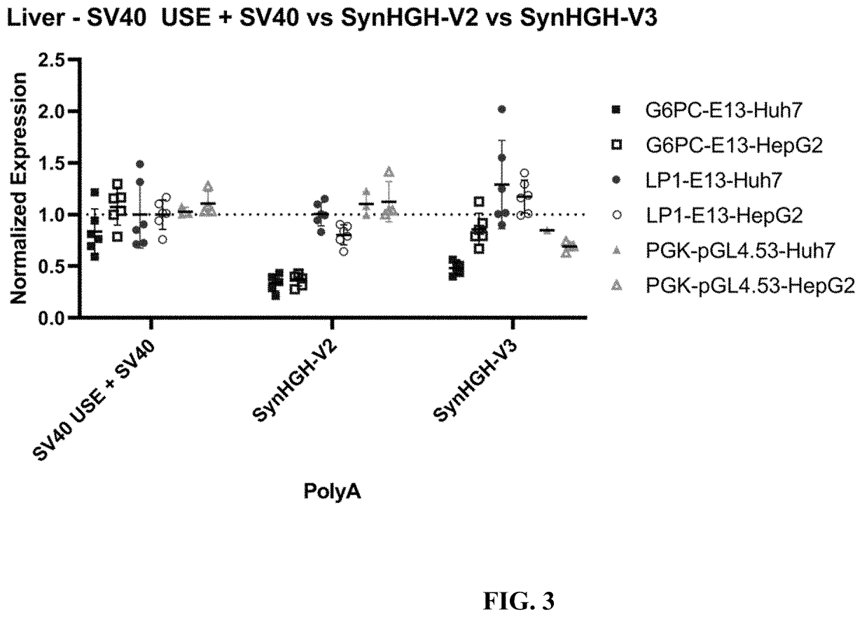

As shown in FIG. 2 and FIG. 3, SynHGH V2 and SynHGH V3 increased gene expression compared to the SV40 polyA sequence.

### Example 3. Construction of Non-Naturally Occurring PolyA SynHGH V2 and SynHGH V3 with Terminator(s)

The SynHGH V2 and SynHGH V3 non-naturally occurring polyAs described in Example 1 were further modified to incorporate one or more terminator sequences. Cloning of these plasmids used the same basic method as described in Example 1. Briefly, a linear vector was created by PCR, the vector and insert assembled via Gibson assembly, using homologous Gibson tags sequences on the insert to drive the assembly. A luciferase-expressing plasmid driven by the LP1 promoter was used as described above. WPRE and C2 double-stranded DNA fragments were obtained and Gibson tags added to the fragments by PCR with primers having 5′ Gibson tag overhangs.

For the WPRE-SynHGH-V2/V3 constructs, the plasmid was linearized by PCR upstream of the synthetic polyA sequence. The WPRE sequence containing 5′ and 3′ Gibson tags was inserted via Gibson assembly. The same method was followed for the SynHGH-V2/V3-C2 constructs, but the linearization of the plasmid was done downstream of the synthetic polyA sequence, as C2 is located downstream of the polyA whereas WPRE is located upstream of the polyA.

The SynHGH-V2/V3 constructs containing both C2 and WPRE required 1) the plasmid minus the entire synthetic polyA sequence, 2) the WPRE sequence with Gibson tags, 3) the synthetic poly sequence, and 4) the C2 sequence with Gibson tags, to be generated by PCR and assembled. As described in Example 1, once the plasmids were assembled, they were transformed into competent cells, plated, colonies picked, miniprepped, and screened for sequence fidelity by Sanger sequencing.

The nucleic acid sequences of the modified SynHGH V2 and SynHGH V3 polyA sequences constructed are detailed in Table 12.

TABLE 12
Modified SynHGH V2 and SynHGH V3 Non-
naturally occurring PolyAs

| | Ele- | | |
|---|---|---|---|
| | ments | | SEQ |
| | 5′ to | | ID |
| Name | 3′ | Nucleic Acid Sequence | NO |
| WPRE- | WPRE | AATCAACCTCTGGATTACAAAATTTGTGAAA | 10 |
| SynHGH | SynHGH | GATTGACTGGTATTCTTAACTATGTTGCTCC | |
| V2 | V2 | TTTTACGCTTGGTGGATACGCTGCTTTACGG | |
| | | CCTTTGTATCTGGCTATTGCTTCCCGTATGG | |
| | | CTTTCATTTTCTCCTCCTTGTATAAATCCTG | |
| | | GTTGCTGTCTCTTTTGGAGGAGTTGTGGCCC | |
| | | GTTGTCAGGCAACGTGGCGTGGTGTGCACTG | |
| | | TGTTTGCTGACGCAACCCCCACTGGTTGGGG | |
| | | CATTGCCACCACCTGTCAGCTCCTTTCCGGG | |
| | | ACTTTCGCTTTCCCCCTCCCTATTGCCACGG | |
| | | CGGAACTCATCGCCGCCTGCCTTGCCCGCTG | |
| | | CTGGACAGGGGCTCGGCTGTTGGGCACTGAC | |
| | | AATTCCGTGGTGTTGTCCCTCTCCTGGCCCT | |
| | | GGAAGTTGCCACTCCAGTGCCGACCAGCCTT | |
| | | GTCCTAATAAACAAGTTAACAACAATTTTGT | |
| | | CTCGTGTGTTGGAATTTTTTGTGTCTCTGGG | |
| | | GTGGAGGGGGGTGGTATGGAGCAAGGGG | |
| SynHGH | SynHGH | CCTCTCCTGGCCCTGGAAGTTGCCACTCCAG | 11 |
| V2-C2 | V2 | TGCCGACCAGCCTTGTCCTAATAAACAAGTT | |
| | C2 | AACAACAATTTTGTCTCGTGTGTTGGAATTT | |
| | | TTTGTGTCTCTGGGGTGGAGGGGGGTGGTAT | |
| | | GGAGCAAGGGGCAGTGCCTCTATCTGGAGGC | |
| | | CAGGTAGGGCTGGCCTTGGGGGAGGGGGAGG | |
| | | CCAGAATGACTCCAAGAGCTACAGGAAGGCA | |
| | | GGTCAGAGACCCCACTGGACAAACAGTGGCT | |
| | | GGACTCTGCACCATAACACACAATCAACAGG | |
| | | GGAGTGAGCTGG | |
| WPRE- | WPRE | AATCAACCTCTGGATTACAAAATTTGTGAAA | 12 |
| SynHGH | SynHGH | GATTGACTGGTATTCTTAACTATGTTGCTCC | |
| V2-C2 | V2 | TTTTACGCTTGGTGGATACGCTGCTTTACGG | |
| | C2 | CCTTTGTATCTGGCTATTGCTTCCCGTATGG | |
| | | CTTTCATTTTCTCCTCCTTGTATAAATCCTG | |
| | | GTTGCTGTCTCTTTTGGAGGAGTTGTGGCCC | |
| | | GTTGTCAGGCAACGTGGCGTGGTGTGCACTG | |
| | | TGTTTGCTGACGCAACCCCCACTGGTTGGGG | |
| | | CATTGCCACCACCTGTCAGCTCCTTTCCGGG | |
| | | ACTTTCGCTTTCCCCCTCCCTATTGCCACGG | |
| | | CGGAACTCATCGCCGCCTGCCTTGCCCGCTG | |
| | | CTGGACAGGGGCTCGGCTGTTGGGCACTGAC | |
| | | AATTCCGTGGTGTTGTCCCTCTCCTGGCCCT | |
| | | GGAAGTTGCCACTCCAGTGCCGACCAGCCTT | |
| | | GTCCTAATAAACAAGTTAACAACAATTTTGT | |
| | | CTCGTGTGTTGGAATTTTTTGTGTCTCTGGG | |
| | | GTGGAGGGGGGTGGTATGGAGCAAGGGG | |
| WPRE- | WPRE | AATCAACCTCTGGATTACAAAATTTGTGAAA | 19 |
| SynHGH | SynHGH | GATTGACTGGTATTCTTAACTATGTTGCTCC | |
| V3 | V3 | TTTTACGCTTGGTGGATACGCTGCTTTACGG | |
| | | CCTTTGTATCTGGCTATTGCTTCCCGTATGG | |
| | | CTTTCATTTTCTCCTCCTTGTATAAATCCTG | |
| | | GTTGCTGTCTCTTTTGGAGGAGTTGTGGCCC | |
| | | GTTGTCAGGCAACGTGGCGTGGTGTGCACTG | |
| | | TGTTTGCTGACGCAACCCCCACTGGTTGGGG | |
| | | CATTGCCACCACCTGTCAGCTCCTTTCCGGG | |
| | | ACTTTCGCTTTCCCCCTCCCTATTGCCACGG | |
| | | CGGAACTCATCGCCGCCTGCCTTGCCCGCTG | |
| | | CTGGACAGGGGCTCGGCTGTTGGGCACTGAC | |
| | | AATTCCGTGGTGTTGTCTTTATTTGTGAAAT | |
| | | TTGTGATGCTATTGCTTTATTTGTAACCATT | |
| | | TTATTTGTGAAATTTGTGATGCTATTGCTTT | |
| | | ATTTGTAACCACAATAAAATTAAGTTGCATC | |
| | | ATTTTGTCTGACTAGGTGTCCTTCTATAATA | |
| | | TTATGGGGTGGAGGGGGGTGGTATGGAGCAA | |
| | | GGGG | |
| SynHGH | SynHGH | TTTATTTGTGAAATTTGTGATGCTATTGCTT | 20 |
| V3-C2 | V3 | TATTTGTAACCATTTTATTTGTGAAATTTGT | |
| | C2 | GATGCTATTGCTTTATTTGTAACCACAATAA | |
| | | AATTAAGTTGCATCATTTTGTCTGACTAGGT | |
| | | GTCCTTCTATAATATTATGGGGTGGAGGGGG | |
| | | GTGGTATGGAGCAAGGGGCAGTGCCTCTATC | |
| | | TGGAGGCCAGGTAGGGCTGGCCTTGGGGGAG | |
| | | GGGGAGGCCAGAATGACTCCAAGAGCTACAG | |
| | | GAAGGCAGGTCAGAGACCCCACTGGACAAAC | |
| | | AGTGGCTGGACTCTGCACCATAACACACAAT | |
| | | CAACAGGGGAGTGAGCTGG | |
| WPRE- | WPRE | AATCAACCTCTGGATTACAAAATTTGTGAAA | 21 |
| SynHGH | SynHGH | GATTGACTGGTATTCTTAACTATGTTGCTCC | |
| V3-C2 | V3 | TTTTACGCTTGGTGGATACGCTGCTTTACGG | |
| | C2 | CCTTTGTATCTGGCTATTGCTTCCCGTATGG | |
| | | CTTTCATTTTCTCCTCCTTGTATAAATCCTG | |
| | | GTTGCTGTCTCTTTTGGAGGAGTTGTGGCCC | |
| | | GTTGTCAGGCAACGTGGCGTGGTGTGCACTG | |
| | | TGTTTGCTGACGCAACCCCCACTGGTTGGGG | |
| | | CATTGCCACCACCTGTCAGCTCCTTTCCGGG | |
| | | ACTTTCGCTTTCCCCCTCCCTATTGCCACGG | |
| | | CGGAACTCATCGCCGCCTGCCTTGCCCGCTG | |
| | | CTGGACAGGGGCTCGGCTGTTGGGCACTGAC | |
| | | AATTCCGTGGTGTTGTCTTTATTTGTGAAAT | |
| | | TTGTGATGCTATTGCTTTATTTGTAACCATT | |
| | | TTATTTGTGAAATTTGTGATGCTATTGCTTT | |
| | | ATTTGTAACCACAATAAAATTAAGTTGCATC | |
| | | ATTTTGTCTGACTAGGTGTCCTTCTATAATA | |
| | | TTATGGGGTGGAGGGGGGTGGTATGGAGCAA | |
| | | GGGGCAGTGCCTCTATCTGGAGGCCAGGTAG | |
| | | GGCTGGCCTTGGGGGAGGGGGAGGCCAGAAT | |
| | | GACTCCAAGAGCTACAGGAAGGCAGGTCAGA | |
| | | GACCCCACTGGACAAACAGTGGCTGGACTCT | |
| | | GCACCATAACACACAATCAACAGGGGAGTGA | |
| | | GCTGG | |

An additional set of non-naturally occurring polyA sequences were made, which incorporated the SV40 polyA sequence with a C2 terminator, a sWPRE (safety modified) terminator, an alpha 2 globin terminator, a human beta globin CoTC terminator, a mouse beta-major globin terminator, or both a C2 terminator and a sWPRE terminator. The modified SV40 polyA sequences constructed are detailed in Table 13.

TABLE 13

| Modified SV40 Non-naturally occurring PolyAs | | | |
|---|---|---|---|
| | | | SEQ |
| | | | ID |
| Name | Elements | Nucleic Acid Sequence | NO |
| SV40 | SV40 | GATCCAGACATGATAAGATACATTGATGAGTTT | 22 |
| | | GGACAAACCACAACTAGAATGCAGTGAAAAAAA | |
| | | TGCTTTATTTGTGAAATTTGTGATGCTATTGCT | |
| | | TTATTTGTAACCATTATAAGCTGCAATAAACAA | |
| | | GTTAACAACAACAATTGCATTCATTTTATGTTT | |
| | | CAGGTTCAGGGGGAGGTGTGGGAGGTTTTTTAA | |
| SV40- | SV40 | GATCCAGACATGATAAGATACATTGATGAGTTT | 23 |
| C2 | C2 | GGACAAACCACAACTAGAATGCAGTGAAAAAAA | |
| | | TGCTTTATTTGTGAAATTTGTGATGCTATTGCT | |
| | | TTATTTGTAACCATTATAAGCTGCAATAAACAA | |
| | | GTTAACAACAACAATTGCATTCATTTTATGTTT | |
| | | CAGGTTCAGGGGGAGGTGTGGGAGGTTTTTTAA | |
| | | aCAGTGCCTCTATCTGGAGGCCAGGTAGGGCTG | |
| | | GCCTTGGGGGAGGGGGAGGCCAGAATGACTCCA | |
| | | AGAGCTACAGGAAGGCAGGTCAGAGACCCCACT | |
| | | GGACAAACAGTGGCTGGACTCTGCACCATAACA | |
| | | CACAATCAACAGGGGAGTGAGCTGG | |
| SV40- | SV40 | AATCAACCTCTGGATTACAAAATTTGTGAAAGA | 24 |
| sWPRE | sWPRE | TTGACTGGTATTCTTAACTATGTTGCTCCTTTT | |
| | | ACGCTtgGTGGATACGCTGCTTTAcgGCCTTTG | |
| | | TATCtgGCTATTGCTTCCCGTATGGCTTTCATT | |
| | | TTCTCCTCCTTGTATAAATCCTGGTTGCTGTCT | |
| | | CTTTtgGAGGAGTTGTGGCCCGTTGTCAGGCAA | |
| | | CGTGGCGTGGTGTGCACTGTGTTTGCTGACGCA | |
| | | ACCCCCACTGGTTGGGGCATTGCCACCACCTGT | |
| | | CAGCTCCTTTCCGGGACTTTCGCTTTCCCCCTC | |
| | | CCTATTGCCACGGCGGAACTCATCGCCGCCTGC | |
| | | CTTGCCCGCTGCTGGACAGGGGCTCGGCTGTTG | |
| | | GGCACTGACAATTCCGTGGTGTTGTCGATCCAG | |
| | | ACATGATAAGATACATTGATGAGTTTGGACAAA | |
| | | CCACAACTAGAATGCAGTGAAAAAAATGCTTTA | |
| | | TTTGTGAAATTTGTGATGCTATTGCTTTATTTG | |
| | | TAACCATTATAAGCTGCAATAAACAAGTTAACA | |
| | | ACAACAATTGCATTCATTTTATGTTTCAGGTTC | |
| | | AGGGGGAGGTGTGGGAGGTTTTTTAA | |
| SV40- | SV40 | GATCCAGACATGATAAGATACATTGATGAGTTT | 25 |
| alpha | alpha 2 | GGACAAACCACAACTAGAATGCAGTGAAAAAAA | |
| 2 | globin | TGCTTTATTTGTGAAATTTGTGATGCTATTGCT | |
| globin | | TTATTTGTAACCATTATAAGCTGCAATAAACAA | |
| | | GTTAACAACAACAATTGCATTCATTTTATGTTT | |
| | | CAGGTTCAGGGGGAGGTGTGGGAGGTTTTTTAA | |
| | | aAACATACGCTCTCCATCAAAACAAAACGAAAC | |
| | | AAAACAAACTAGCAAAATAGGCTGTCCCCAGTG | |
| | | CAAGTGCAGGTGCCAGAACATTTCTCT | |
| SV40- | SV40 | GATCCAGACATGATAAGATACATTGATGAGTTT | 26 |
| human | human | GGACAAACCACAACTAGAATGCAGTGAAAAAAA | |
| beta | beta | TGCTTTATTTGTGAAATTTGTGATGCTATTGCT | |
| globin | globin | TTATTTGTAACCATTATAAGCTGCAATAAACAA | |
| CoTC | CoTC | GTTAACAACAACAATTGCATTCATTTTATGTTT | |
| | | CAGGTTCAGGGGGAGGTGTGGGAGGTTTTTTAA | |
| | | aCAATAACAAACAAAAAATTAAAAATAGGAAAA | |
| | | TAAAAAAATTAAAAAGAAGAAAATCCTGCCATT | |
| | | TATGCGAGAATTGATGAACCTGGAGGATGTAAA | |
| | | ACTAAGAAAAATAAGCCTGACACAAAAAGACAA | |
| | | ATACTACACAACCTTGCTCATATGTGAAACATA | |
| | | AAAAAGTCACTCTCATGGAAACAGACAGTAGAG | |
| | | GTATGGTTTCCAGGGGTTGGGGGTGGGAGAATC | |
| | | AGGAAACTATTACTCAAAGGGTATAAAATTTCA | |
| | | GTTATGTGGGATGAATAAATT | |
| SV40- | SV40 | GATCCAGACATGATAAGATACATTGATGAGTTT | 27 |
| Mouse | Mouse | GGACAAACCACAACTAGAATGCAGTGAAAAAAA | |
| beta- | beta- | TGCTTTATTTGTGAAATTTGTGATGCTATTGCT | |
| major | major | TTATTTGTAACCATTATAAGCTGCAATAAACAA | |
| globin | globin | GTTAACAACAACAATTGCATTCATTTTATGTTT | |
| | | CAGGTTCAGGGGGAGGTGTGGGAGGTTTTTTAA | |
| | | aGAAGTAAAGAGTTAGAGTATGGTGAGAAATTA | |
| | | TAAACCATCAAAGAAAAAAATACAGGACCCATA | |
| | | AAGG | |
| WPRE- | WPRE | AATCAACCTCTGGATTACAAAATTTGTGAAAGA | 28 |
| SV40- | SV40 | TTGACTGGTATTCTTAACTATGTTGCTCCTTTT | |
| C2 | C2 | ACGCTtgGTGGATACGCTGCTTTAcgGCCTTTG | |
| | | TATCtgGCTATTGCTTCCCGTATGGCTTTCATT | |
| | | TTCTCCTCCTTGTATAAATCCTGGTTGCTGTCT | |
| | | CTTTtgGAGGAGTTGTGGCCCGTTGTCAGGCAA | |
| | | CGTGGCGTGGTGTGCACTGTGTTTGCTGACGCA | |
| | | ACCCCCACTGGTTGGGGCATTGCCACCACCTGT | |
| | | CAGCTCCTTTCCGGGACTTTCGCTTTCCCCCTC | |
| | | CCTATTGCCACGGCGGAACTCATCGCCGCCTGC | |
| | | CTTGCCCGCTGCTGGACAGGGGCTCGGCTGTTG | |
| | | GGCACTGACAATTCCGTGGTGTTGTCGATCCAG | |
| | | ACATGATAAGATACATTGATGAGTTTGGACAAA | |
| | | CCACAACTAGAATGCAGTGAAAAAAATGCTTTA | |
| | | TTTGTGAAATTTGTGATGCTATTGCTTTATTTG | |
| | | TAACCATTATAAGCTGCAATAAACAAGTTAACA | |
| | | ACAACAATTGCATTCATTTTATGTTTCAGGTTC | |
| | | AGGGGGAGGTGTGGGAGGTTTTTTAAaCAGTGC | |
| | | CTCTATCTGGAGGCCAGGTAGGGCTGGCCTTGG | |
| | | GGGAGGGGGAGGCCAGAATGACTCCAAGAGCTA | |
| | | CAGGAAGGCAGGTCAGAGACCCCACTGGACAAA | |
| | | CAGTGGCTGGACTCTGCACCATAACACACAATC | |
| | | AACAGGGGAGTGAGCTGG | |

### Example 4. Evaluation of Gene Expression Using Vectors with Terminator(s)

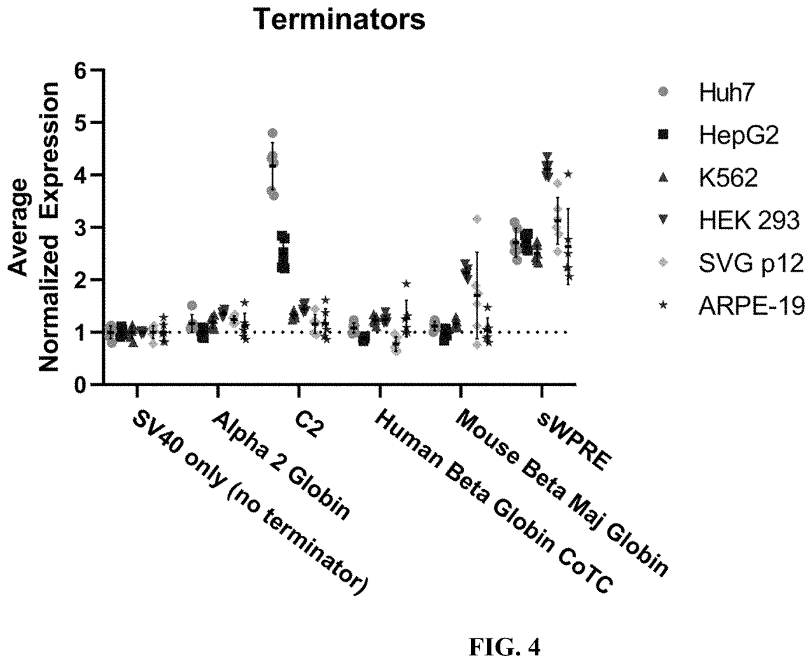

The SV40 terminator non-naturally occurring polyA sequences described in Table 13 were incorporated into a gene expression vector encoding a luciferase reporter protein and a PGK promoter (according to methods described in Example 2). The vectors were introduced into cultured cells (Huh7, HepG2, K562, HEK 293, SVG p12, ARPE-19) and expression of the luciferase reporter protein analyzed (according to methods described in Example 2). As shown in FIG. 4, inclusion of a terminator, particularly C2 or WPRE, increased protein expression compared to the no-terminator control SV40 polyA sequence.

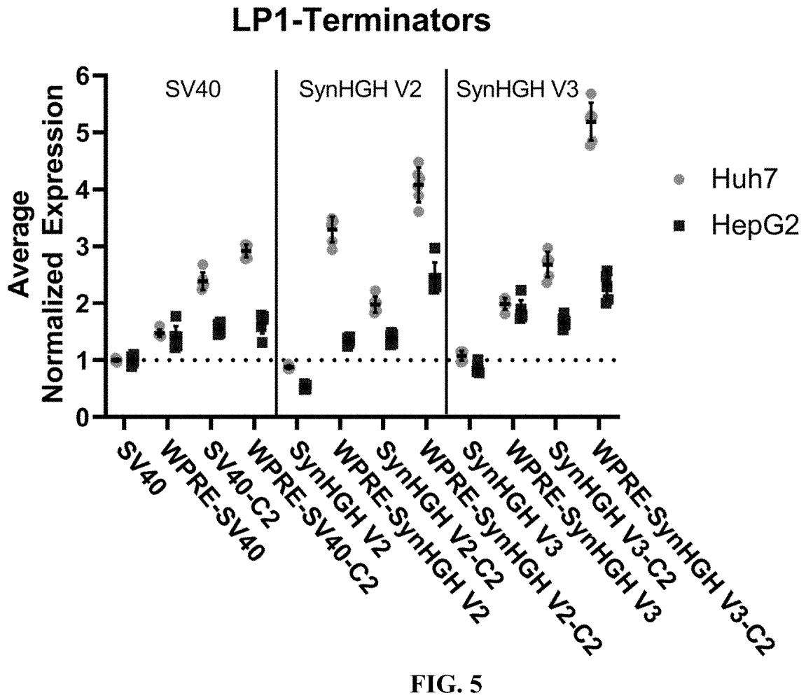

The SynHGH V2 and SynHGH V3 non-naturally occurring polyAs described in Table 12 were incorporated into a gene expression vector encoding a luciferase reporter protein and a promoter (PGK or LP1) (according to methods described in Example 2). The vectors were introduced into cultured cells (Huh7, HepG2, K562, HEK 293, SVG p12, ARPE-19) and expression of the luciferase reporter protein analyzed (according to methods described in Example 2). As shown in FIG. 5, inclusion of a terminator, particularly WPRE-SynHGH V2-C2 and WPRE-SynHGH V3-C2, increased protein expression compared to the no-terminator controls.

1. A polynucleotide comprising a non-naturally occurring polyadenylation (polyA) sequence, said polynucleotide comprising from 5′ to 3′:
a. a polyA signal that comprises the nucleic acid sequence set forth in SEQ ID NO: 1;
b. a first intervening nucleic acid sequence derived from a naturally occurring polyA sequence of a first gene, wherein said naturally occurring polyA sequence of a first gene comprises a polyA signal, a GT rich region, and a nucleic acid sequence positioned between said polyA signal and said GT rich region,
i. wherein said first intervening nucleic acid sequence comprises a sequence of at least 10 nucleotides in length that is derived from said nucleic acid sequence positioned between said polyA signal and said GT rich region of said naturally occurring polyA sequence of a first gene, and
ii. wherein said first intervening nucleic acid sequence comprises 0, 1, 2, or 3 nucleotide modifications relative to the corresponding region of said naturally occurring polyA sequence of a first gene; and
c. a first GT rich nucleic acid sequence derived from a naturally occurring polyA sequence of a second gene, wherein said naturally occurring polyA sequence of a second gene comprises a polyA signal and a GT rich region;
i. wherein said first GT rich nucleic acid sequence comprises a sequence of at least 5 nucleotides in length that is derived from said GT rich region of said naturally occurring polyA sequence of a second gene,
ii. wherein said first GT rich nucleic acid sequence comprises 0, 1, or 2 nucleotide modifications relative to the corresponding region of said naturally occurring polyA sequence of a second gene, and
iii. wherein said first GT rich nucleic acid sequence is positioned 10-30 nucleotides downstream of said polyA signal that comprises the nucleic acid sequence set forth in SEQ ID NO: 1; and
wherein said first gene and said second gene are different.

2. The polynucleotide of claim 1, wherein:
said first gene is a human or non-human gene, optionally wherein the non-human gene is selected from the group consisting of a viral, bacterial, or non-human mammalian gene, optionally wherein said viral gene is a simian virus 40 (SV40) late gene;
said first intervening nucleic acid sequence derived from a naturally occurring polyA sequence of a first gene comprises the nucleic acid sequence set forth in SEQ ID NO: 4;
said first GT rich nucleic acid sequence derived from a naturally occurring polyA sequence of a second gene comprises the nucleic acid sequence set forth in SEQ ID NO: 2;
said first GT rich nucleic acid sequence derived from a naturally occurring polyA sequence of a second gene is positioned 15-30 nucleotides downstream of said polyA signal that comprises the nucleic acid sequence set forth in SEQ ID NO: 1;
said second gene is a human or non-human gene, optionally wherein said human gene is human growth hormone (HGH); and/or
said polynucleotide is no more than 300, 250, or 200 nucleotides in length.

3.-13. (canceled)

14. The polynucleotide of claim 1, further comprising a second GT rich nucleic acid sequence derived from a naturally occurring polyA sequence of a third gene, wherein said naturally occurring polyA sequence of a third gene comprises a polyA signal and a GT rich region;
a. wherein said second GT rich nucleic acid sequence comprises a nucleic acid sequence of at least 5 nucleotides in length that is derived from said GT rich region of said naturally occurring polyA sequence of a third gene;
b. wherein said second GT rich nucleic acid sequence comprises 0, 1, or 2 nucleotide modifications relative to the corresponding region of said naturally occurring polyA sequence of a third gene; and
c. wherein said second GT rich nucleic acid sequence is positioned 5-100 nucleotides downstream of said first GT rich nucleic acid sequence,
optionally wherein:
said second GT rich nucleic acid sequence derived from a naturally occurring polyA sequence of a third gene comprises the nucleic acid sequence set forth in SEQ ID NO: 3;
said third gene is a human or non-human gene, optionally wherein said human gene is HGH, and/or
said third gene and said second gene are the same or different.

15.-20. (canceled)

21. The polynucleotide of claim 14, further comprising a second intervening nucleic acid sequence derived from a naturally occurring polyA sequence of a fourth gene, wherein said naturally occurring polyA sequence of a fourth gene comprises a first GT rich region, a second GT rich region, and a nucleic acid sequence positioned between said first GT rich region and said second GT rich region,
a. wherein said second intervening nucleic acid sequence comprises a sequence of at least 5 nucleotides in length that is derived from said nucleic acid sequence positioned between said first GT rich region and said second GT rich region of said naturally occurring polyA sequence of a fourth gene, and
b. wherein said second intervening nucleic acid sequence comprises 0, 1, 2, or 3 nucleotide modifications relative to the corresponding region of said naturally occurring polyA sequence of a fourth gene,
optionally wherein:
said fourth gene is a human gene or non-human gene, optionally wherein said non-human gene is a viral, bacterial, or non-human mammalian gene, optionally wherein said non-human mammalian gene is bovine growth hormone (BGH) or rabbit beta globin (RBG);
said fourth gene and said first gene are the same or different said second intervening nucleic acid sequence derived from a naturally occurring polyA sequence of a fourth gene comprises the nucleic acid sequence set forth in SEQ ID NO: 5; and/or
said second intervening nucleic acid sequence derived from a naturally occurring polyA sequence of a fourth gene is positioned downstream of said first GT rich nucleic acid sequence and upstream of said second GT rich nucleic acid sequence.

22.-31. (canceled)

32. The polynucleotide of claim 1, further comprising an upstream sequence element derived from a naturally occurring polyA sequence of a fifth gene, wherein said naturally occurring polyA sequence of a fifth gene comprises a polyA signal, a GT rich region, and a nucleic acid sequence positioned immediately upstream of said polyA signal; and wherein said upstream sequence element comprises 1-100 nucleotides derived from said nucleic acid sequence positioned immediately upstream of said polyA signal of said naturally occurring polyA sequence of a fifth gene, optionally wherein said fifth gene is selected from a human or non-human gene.

33.-34. (canceled)

35. The polynucleotide of claim 1, wherein said polynucleotide comprises a sequence with at least 95%, 96%, 97%, 98%, 99%, or 100% identity to the sequence set forth in SEQ ID NO: 7.

36. (canceled)

37. The polynucleotide of claim 1, further comprising a first terminator positioned upstream or downstream of said polyA signal that comprises the nucleic acid sequence set forth in SEQ ID NO: 1, optionally wherein:
said first terminator is selected from the group consisting of a woodchuck hepatitis virus posttranscriptional regulatory element (WPRE), a human C2 pause site element, a SV40 upstream sequence element, an alpha 2 globin pause site element, a human beta globin cotranscriptional cleavage (CoTC) sequence element, and a mouse beta-major globin pause site element;
said first terminator comprises
a. a human C2 gene pause site element, wherein said human C2 gene pause site element is positioned downstream of said polyA signal that comprises the nucleic acid sequence set forth in SEQ ID NO: 1, or
b. a WPRE, wherein said WPRE is positioned upstream of said polyA signal that comprises the nucleic acid sequence set forth in SEQ ID NO: 1, and/or
said first terminator comprises a nucleic acid sequence of at least 95%, 96%, 97%, 98%, 99%, or 100% identity to the sequence set forth in SEQ ID NO: 8 or 9.

38.-40. (canceled)

41. The polynucleotide of claim 37, wherein said polynucleotide comprises a second terminator, optionally wherein:
said first and said second terminator are different;
said first terminator comprises a human C2 gene pause site element, wherein said human C2 gene pause site element is positioned downstream of said polyA signal that comprises the nucleic acid sequence set forth in SEQ ID NO: 1,
said second terminator comprises a WPRE, wherein said WPRE is positioned upstream of said polyA signal that comprises the nucleic acid sequence set forth in SEQ ID NO: 1, and/or
said first terminator comprises a nucleic acid sequence of at least 95%, 96%, 97%, 98%, 99%, or 100% identity to the sequence set forth in SEQ ID NO: 8, and said second terminator comprises a nucleic acid sequence of at least 95%, 96%, 97%, 98%, 99%, or 100% identity to the sequence set forth in SEQ ID NO: 9.

42.-44. (canceled)

45. A polynucleotide comprising a non-naturally occurring polyadenylation (polyA) sequence, said polynucleotide comprising from 5′ to 3′:
a. an upstream sequence element nucleic acid sequence derived from a naturally occurring polyA sequence of a first gene, wherein said naturally occurring polyA sequence of a first gene comprises a naturally occurring upstream sequence element, a polyA signal, and a GT rich region,
i. wherein said upstream sequence element comprises a functional nucleic acid sequence of said naturally occurring upstream sequence element of said naturally occurring polyA sequence of a first gene, and
ii. wherein said upstream sequence element nucleic acid sequence comprises 0, 1, 2, or 3 nucleotide modifications relative to the corresponding region of said naturally occurring polyA sequence of a first gene;
b. a polyA signal that comprises the nucleic acid sequence set forth in SEQ ID NO: 1;
c. a first GT rich nucleic acid sequence derived from a naturally occurring polyA sequence of a second gene, wherein said naturally occurring polyA sequence of a second gene comprises a polyA signal and a GT rich region;
i. wherein said first GT rich nucleic acid sequence comprises a sequence of at least 5 nucleotides in length that is derived from said GT rich region of said naturally occurring polyA sequence of a second gene,
ii. wherein said first GT rich nucleic acid sequence comprises 0, 1, or 2 nucleotide modifications relative to the corresponding region of said naturally occurring polyA sequence of a second gene, and
iii. wherein said first GT rich nucleic acid sequence is positioned 10-30 nucleotides downstream of said polyA signal that comprises the nucleic acid sequence set forth in SEQ ID NO: 1; and
wherein said first gene and said second gene are different.

46. The polynucleotide of claim 45, wherein:
said first gene is a human or non-human gene, optionally wherein the non-human gene is selected from the group consisting of a viral, bacterial, or non-human mammalian gene, optionally wherein said viral gene is simian virus 40 (SV40) late gene;
said second gene is a human or non-human gene, optionally wherein the human gene is HGH;
said upstream sequence element nucleic acid sequence comprises the nucleic acid sequence set forth in SEQ ID NO: 13 or 15;
said upstream sequence element nucleic acid sequence is positioned immediately upstream of said polyA signal that comprises the nucleic acid sequence set forth in SEQ ID NO: 1, optionally wherein said polynucleotide comprises at least two copies of said upstream sequence element nucleic acid sequence, optionally wherein said two copies of said upstream sequence element nucleic acid sequence are consecutively positioned upstream of said polyA signal that comprises the nucleic acid sequence set forth in SEQ ID NO: 1;
said first GT rich nucleic acid sequence derived from a naturally occurring polyA sequence of a second gene comprises the nucleic acid sequence set forth in SEQ ID NO: 2;
said first GT rich nucleic acid sequence derived from a naturally occurring polyA sequence of a second gene is positioned 15-30 nucleotides downstream of said polyA signal that comprises the nucleic acid sequence set forth in SEQ ID NO: 1;
said polynucleotide comprises a sequence with at least 95%, 96%, 97%, 98%, 99%, or 100% identity to the sequence set forth in SEQ ID NO: 18; and/or
said polynucleotide is no more than 300, 250, or 200 nucleotides in length.

47.-61. (canceled)

62. The polynucleotide of claim 45, further comprising a second GT rich nucleic acid sequence derived from a naturally occurring polyA sequence of a third gene, wherein said naturally occurring polyA sequence of a third gene comprises a polyA signal, a first GT rich region, and a second GT rich region;
a. wherein said second GT rich nucleic acid sequence comprises a sequence of at least 5 nucleotides in length that is derived from said second GT rich region of said naturally occurring polyA sequence of a third gene,
b. wherein said second GT rich nucleic acid sequence comprises 0, 1, or 2 nucleotide modifications relative to the corresponding region of said naturally occurring polyA sequence of a third gene; and
c. wherein said second GT rich nucleic acid region is positioned 5-100 nucleotides downstream of said first GT rich nucleic acid sequence,
optionally wherein:
said second GT rich nucleic acid sequence derived from said naturally occurring polyA sequence of a third gene comprises the nucleic acid sequence set forth in SEQ ID NO: 3;
said third gene is a human gene or non-human gene, optionally wherein said human gene is HGH; and/or
said third gene and said second gene are the same or different.

63.-71. (canceled)

72. The polynucleotide of claim 45, further comprising a first terminator positioned upstream or downstream of said polyA signal that comprises the nucleic acid sequence set forth in SEQ ID NO: 1, optionally wherein:
said first terminator is selected from the group consisting of a WPRE, a human C2 pause site element, a SV40 upstream sequence element, an alpha 2 globin pause site element, a human beta globin CoTC element, and a mouse beta-major globin pause site element;
said first terminator comprises
a. a human C2 gene pause site element, wherein said human C2 gene pause site element is positioned downstream of said polyA signal that comprises the nucleic acid sequence set forth in SEQ ID NO: 1, or
b. a WPRE, wherein said WPRE is positioned upstream of said polyA signal that comprises the nucleic acid sequence set forth in SEQ ID NO: 1, and/or
said first terminator comprises a nucleic acid sequence of at least 95%, 96%, 97%, 98%, 99%, or 100% identity to the sequence set forth in SEQ ID NO: 8 or 9.

73.-75. (canceled)

76. The polynucleotide of claim 72, wherein said polynucleotide comprises a second terminator, optionally wherein:
said first and said second terminator are different;
said first terminator comprises a human C2 gene pause site element, wherein said human C2 gene pause site element is positioned downstream of said polyA signal that comprises the nucleic acid sequence set forth in SEQ ID NO: 1,
said second terminator comprises a WPRE, wherein said WPRE is positioned upstream of said polyA signal that comprises the nucleic acid sequence set forth in SEQ ID NO: 1, and/or
said first terminator comprises a nucleic acid sequence of at least 95%, 96%, 97%, 98%, 99%, or 100% identity to the sequence set forth in SEQ ID NO: 8, and said second terminator comprises a nucleic acid sequence of at least 95%, 96%, 97%, 98%, 99%, or 100% identity to the sequence set forth in SEQ ID NO: 9.

77.-79. (canceled)

80. The polynucleotide of claim 1, wherein upon inclusion in a suitable gene expression cassette, said polyA sequence mediates comparable or increased of a gene in said gene expression cassette relative to a control gene expression cassette that comprises a control polyA sequence, optionally wherein said polyA sequence mediates at least a 2-fold, 3-fold, 4-fold, or 5-fold increase in expression of a gene in said gene expression relative to a control gene expression cassette that comprises a control polyA sequence.

81. (canceled)

82. The polynucleotide of claim 1, wherein:
said polynucleotide does not contain a human miRNA binding site;
said polynucleotide is a DNA polynucleotide; or
said polynucleotide is an RNA polynucleotide.

83. (canceled)

84. A polynucleotide that is the complement of the polynucleotide of claim 1.

85. (canceled)

86. A polynucleotide comprising a terminator that comprises a nucleic acid sequence of at least 85%, 86%, 87%, 88%, 89%, 90%, 91%, 92%, 93%, 94%, 95%, 96%, 97%, 98%, 99%, or 100% identity to the sequence set forth in SEQ ID NO: 9.

87. A vector comprising:
a. a transgene that encodes a target protein; and
b. the polynucleotide of claim 1,
optionally wherein:
said vector is a viral vector or a non-viral vector, optionally wherein said nonviral vector is a plasmid or said viral vector is an adeno-associated virus (AAV) vector; and/or
upon introduction into a host cell, said vector mediates comparable or increased expression of said gene relative to a control vector comprising a control polyA sequence, optionally wherein said vector mediates increased expression of said gene by at least 2-fold, 3-fold, 4-fold, or 5-fold relative to a control vector comprising a control polyA sequence.

88.-93. (canceled)

94. A method of expressing a transgene in a cell, said method comprising introducing the vector of claim 87 into the cell.

95. A method of modifying a cell, said method comprising introducing the polynucleotide of claim 1 into the cell.

96. A cell comprising the polynucleotide of claim 1.


SEQUENCE LISTING (54 sequences)

SEQ ID NO: 1-2 - skipped (2 entries)

SEQ ID NO: 3 - DNA, 31 bp, synthetic construct
  source 1..31: mol_type = other DNA
ggggtggaggggggtggtatggagcaagggg

SEQ ID NO: 4 - DNA, 14 bp, synthetic construct
  source 1..14: mol_type = other DNA
caagttaacaacaa

SEQ ID NO: 5 - DNA, 26 bp, synthetic construct
  source 1..26: mol_type = other DNA
cgtgtgttggaattttttgtgtctct

SEQ ID NO: 6 - DNA, 50 bp, synthetic construct
  source 1..50: mol_type = other DNA
cctctcctggccctggaagttgccactccagtgccgaccagccttgtcct

SEQ ID NO: 7 - DNA, 135 bp, synthetic construct
  source 1..135: mol_type = other DNA
cctctcctggccctggaagttgccactccagtgccgaccagccttgtcctaataaacaagttaacaacaattttgtctcgtgtgttggaattttttgtgtctctggggtggaggggggtggtatggagcaagggg

SEQ ID NO: 8 - DNA, 156 bp, synthetic construct
  source 1..156: mol_type = other DNA
cagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctgg

SEQ ID NO: 9 - DNA, 389 bp, synthetic construct
  source 1..389: mol_type = other DNA
aatcaacctctggattacaaaatttgtgaaagattgactggtattcttaactatgttgctccttttacgcttggtggatacgctgctttacggcctttgtatctggctattgcttcccgtatggctttcattttctcctccttgtataaatcctggttgctgtctcttttggaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtc

SEQ ID NO: 10 - DNA, 524 bp, synthetic construct
  source 1..524: mol_type = other DNA
aatcaacctctggattacaaaatttgtgaaagattgactggtattcttaactatgttgctccttttacgcttggtggatacgctgctttacggcctttgtatctggctattgcttcccgtatggctttcattttctcctccttgtataaatcctggttgctgtctcttttggaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtccctctcctggccctggaagttgccactccagtgccgaccagccttgtcctaataaacaagttaacaacaattttgtctcgtgtgttggaattttttgtgtctctggggtggaggggggtggtatggagcaagggg

SEQ ID NO: 11 - DNA, 291 bp, synthetic construct
  source 1..291: mol_type = other DNA
cctctcctggccctggaagttgccactccagtgccgaccagccttgtcctaataaacaagttaacaacaattttgtctcgtgtgttggaattttttgtgtctctggggtggaggggggtggtatggagcaaggggcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctgg

SEQ ID NO: 12 - DNA, 524 bp, synthetic construct; identical to SEQ ID NO: 10
  source 1..524: mol_type = other DNA

SEQ ID NO: 13 - DNA, 44 bp, synthetic construct
  source 1..44: mol_type = other DNA
tttatttgtgaaatttgtgatgctattgctttatttgtaaccac

SEQ ID NO: 14 - DNA, 44 bp, synthetic construct
  source 1..44: mol_type = other DNA
tttatttgtgaaatttgtgatgctattgctttatttgtaaccat

SEQ ID NO: 15 - DNA, 88 bp, synthetic construct
  source 1..88: mol_type = other DNA
tttatttgtgaaatttgtgatgctattgctttatttgtaaccattttatttgtgaaatttgtgatgctattgctttatttgtaaccac

SEQ ID NO: 16 - DNA, 88 bp, synthetic construct
  source 1..88: mol_type = other DNA
tttatttgtgaaatttgtgatgctattgctttatttgtaaccattttatttgtgaaatttgtgatgctattgctttatttgtaaccat

SEQ ID NO: 17 - DNA, 79 bp, synthetic construct
  source 1..79: mol_type = other DNA
attaagttgcatcattttgtctgactaggtgtccttctataatattatggggtggaggggggtggtatggagcaagggg

SEQ ID NO: 18 - DNA, 173 bp, synthetic construct
  source 1..173: mol_type = other DNA
tttatttgtgaaatttgtgatgctattgctttatttgtaaccattttatttgtgaaatttgtgatgctattgctttatttgtaaccacaataaaattaagttgcatcattttgtctgactaggtgtccttctataatattatggggtggaggggggtggtatggagcaagggg

SEQ ID NO: 19 - DNA, 562 bp, synthetic construct
  source 1..562: mol_type = other DNA
aatcaacctctggattacaaaatttgtgaaagattgactggtattcttaactatgttgctccttttacgcttggtggatacgctgctttacggcctttgtatctggctattgcttcccgtatggctttcattttctcctccttgtataaatcctggttgctgtctcttttggaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtctttatttgtgaaatttgtgatgctattgctttatttgtaaccattttatttgtgaaatttgtgatgctattgctttatttgtaaccacaataaaattaagttgcatcattttgtctgactaggtgtccttctataatattatggggtggaggggggtggtatggagcaagggg

SEQ ID NO: 20 - DNA, 329 bp, synthetic construct
  source 1..329: mol_type = other DNA
tttatttgtgaaatttgtgatgctattgctttatttgtaaccattttatttgtgaaatttgtgatgctattgctttatttgtaaccacaataaaattaagttgcatcattttgtctgactaggtgtccttctataatattatggggtggaggggggtggtatggagcaaggggcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctgg

SEQ ID NO: 21 - DNA, 718 bp, synthetic construct
  source 1..718: mol_type = other DNA
aatcaacctctggattacaaaatttgtgaaagattgactggtattcttaactatgttgctccttttacgcttggtggatacgctgctttacggcctttgtatctggctattgcttcccgtatggctttcattttctcctccttgtataaatcctggttgctgtctcttttggaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtctttatttgtgaaatttgtgatgctattgctttatttgtaaccattttatttgtgaaatttgtgatgctattgctttatttgtaaccacaataaaattaagttgcatcattttgtctgactaggtgtccttctataatattatggggtggaggggggtggtatggagcaaggggcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctgg

SEQ ID NO: 22 - DNA, 198 bp, synthetic construct
  source 1..198: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 23 - DNA, 355 bp, synthetic construct
  source 1..355: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctgg

SEQ ID NO: 24 - DNA, 587 bp, synthetic construct
  source 1..587: mol_type = other DNA
aatcaacctctggattacaaaatttgtgaaagattgactggtattcttaactatgttgctccttttacgcttggtggatacgctgctttacggcctttgtatctggctattgcttcccgtatggctttcattttctcctccttgtataaatcctggttgctgtctcttttggaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 25 - DNA, 291 bp, synthetic construct
  source 1..291: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaaaacatacgctctccatcaaaacaaaacgaaacaaaacaaactagcaaaataggctgtccccagtgcaagtgcaggtgccagaacatttctct

SEQ ID NO: 26 - DNA, 483 bp, synthetic construct
  source 1..483: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacaataacaaacaaaaaattaaaaataggaaaataaaaaaattaaaaagaagaaaatcctgccatttatgcgagaattgatgaacctggaggatgtaaaactaagaaaaataagcctgacacaaaaagacaaatactacacaaccttgctcatatgtgaaacataaaaaagtcactctcatggaaacagacagtagaggtatggtttccaggggttgggggtgggagaatcaggaaactattactcaaagggtataaaatttcagttatgtgggatgaataaatt

SEQ ID NO: 27 - DNA, 268 bp, synthetic construct
  source 1..268: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagaagtaaagagttagagtatggtgagaaattataaaccatcaaagaaaaaaatacaggacccataaagg

SEQ ID NO: 28 - DNA, 744 bp, synthetic construct
  source 1..744: mol_type = other DNA
aatcaacctctggattacaaaatttgtgaaagattgactggtattcttaactatgttgctccttttacgcttggtggatacgctgctttacggcctttgtatctggctattgcttcccgtatggctttcattttctcctccttgtataaatcctggttgctgtctcttttggaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctgg

SEQ ID NO: 29-42 - skipped (14 entries)

SEQ ID NO: 43 - DNA, 30 bp, synthetic construct
  source 1..30: mol_type = other DNA
aaatcgataaggatccgtcgaccgatgccc

SEQ ID NO: 44 - DNA, 30 bp, synthetic construct
  source 1..30: mol_type = other DNA
ttacacggcgatcttgccgcccttcttggc

SEQ ID NO: 45 - DNA, 25 bp, synthetic construct
  source 1..25: mol_type = other DNA
gaagggcggcaagatcgccgtgtaa

SEQ ID NO: 46 - DNA, 29 bp, synthetic construct
  source 1..29: mol_type = other DNA
aaatcgataaggatccgtcgaccgatgcc

SEQ ID NO: 47 - DNA, 1653 bp, synthetic construct
  source 1..1653: mol_type = other DNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 48 - DNA, 1653 bp, synthetic construct
  source 1..1653: mol_type = other DNA
atggaggatgccaagaatattaagaaaggccctgccccattctaccctctggaagatggcactgctggagagcaactgcacaaggccatgaagtcctatgccctggtccctggcaccattgccttcactgatgctcacattgaggtggacatcacctatgctgaatactttgagatgtctgtgaggctggcagaagccatgaaaagatatggactgaacaccaaccacaggattgtggtgtgctctgagaactctctccagttcttcatgcctgtgttaggagccctgttcattggagtggctgtggcccctgccaatgacatctacaatgagagagagctcctgaacagcatgggcatcagccagccaactgtggtctttgtgagcaagaagggcctgcaaaagatcctgaatgtgcagaagaagctgcccatcatccagaagatcatcatcatggacagcaagactgactaccagggcttccagagcatgtatacctttgtgaccagccacttaccccctggcttcaatgagtatgactttgtgcctgagagctttgacagggacaagaccattgctctgattatgaacagctctggctccactggactgcccaaaggtgtggctctgccccacagaactgcttgtgtgagattcagccatgccagagaccccatctttggcaaccagatcatccctgacactgccatcctgtctgtggttccattccatcatggctttggcatgttcacaacactggggtacctgatctgtggcttcagagtggtgctgatgtataggtttgaggaggagctgtttctgaggagcctacaagactacaagatccagtctgccctgctggtgcccactctgttcagcttctttgccaagagcaccctcattgacaagtatgacctgagcaacctgcatgagattgcctctggaggagcacccctgagcaaggaggtgggtgaggctgtggcaaagaggttccatctcccaggaatcagacagggctatggcctgactgagaccacctctgccatcctcatcacccctgaaggagatgacaagcctggtgctgtgggcaaggtggttcccttttttgaggccaaggtggtggacctggacactggcaagaccctgggagtgaaccagaggggtgagctgtgtgtgaggggtcccatgatcatgtctggctatgtgaacaaccctgaggccaccaatgccctgattgacaaggatggctggctgcactctggtgacattgcctactgggatgaggatgagcactttttcattgtggacaggctgaagagcctcatcaagtacaaaggctaccaagtggcacctgctgagctagagagcatcctgctccagcaccccaacatctttgatgctggtgtggctggcctgcctgatgatgatgctggagagctgcctgctgctgttgtggttctggagcatggaaagaccatgactgagaaggagattgtggactatgtggccagtcaggtgaccactgccaagaagctgaggggaggtgtggtgtttgtggatgaggtgccaaagggtctgactggcaagctggatgccagaaagatcagagagatcctgatcaaggccaagaagggtggcaaaatcgccgtctag

SEQ ID NO: 49 - DNA, 92 bp, synthetic construct
  source 1..92: mol_type = other DNA
aacatacgctctccatcaaaacaaaacgaaacaaaacaaactagcaaaataggctgtccccagtgcaagtgcaggtgccagaacatttctct

SEQ ID NO: 50 - DNA, 284 bp, synthetic construct
  source 1..284: mol_type = other DNA
caataacaaacaaaaaattaaaaataggaaaataaaaaaattaaaaagaagaaaatcctgccatttatgcgagaattgatgaacctggaggatgtaaaactaagaaaaataagcctgacacaaaaagacaaatactacacaaccttgctcatatgtgaaacataaaaaagtcactctcatggaaacagacagtagaggtatggtttccaggggttgggggtgggagaatcaggaaactattactcaaagggtataaaatttcagttatgtgggatgaataaatt

SEQ ID NO: 51 - DNA, 69 bp, synthetic construct
  source 1..69: mol_type = other DNA
gaagtaaagagttagagtatggtgagaaattataaaccatcaaagaaaaaaatacaggacccataaagg

SEQ ID NO: 52 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 53 - DNA, 592 bp, synthetic construct
  source 1..592: mol_type = other DNA
aatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctg

SEQ ID NO: 54 - DNA, 389 bp, synthetic construct
  source 1..389: mol_type = other DNA
aatcaacctctggattacaaaatttgtgaaagattgactgatattcttaactatgttgctccttttacgcttggtggatatgctgctttacggcctctgtatctggctattgcttcccgtacggctttcgttttctcctccttgtataaatcctggttgctgtctcttttggaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc